(19) United States
(12) Patent Application Publication
(10) Pub. No.: US 2003/0074081 A1
(43) Pub. Date: Apr. 17, 2003

(54) Non-uniform porosity tissue implant

(76) Inventor: Reed A. Ayers, Golden, CO (US)

Correspondence Address: Steven C. Petersen, Hogan & Hartson, LLP, Suite 1500, 1200 17th Street, Denver, CO, 80202, US

(21) Appl. No.: 10/199,139

(22) Filed: Jul. 19, 2002

Related U.S. Application Data
(63) Continuation of application No. 09/957,829, filed on Sep. 21, 2001, now abandoned.
(60) Provisional application No. 60/234,841, filed on Sep. 22, 2000.

(51) Int. Cl. A61F 2/28; A61F 2/02

(52) U.S. Cl. 623/23.5; 623/23.76

(57) ABSTRACT

The present invention is generally directed to a method for the production of tissue implants and prosthetics, including but not limited to orthopedic implants and prosthetics which have a controlled and directional gradient of porosity moving through all or one or more portions of the implant, as well as the implants produced by such a method. The non-uniform porosity gradient may be linear or more complex, and is preferably produced to have a continuous gradient within the desired regions. The desired effect is to create an implant which more closely mimics the natural structure of bone, and which improves the quality of the bone growth that occurs within the implant. In addition, implants can be created with varying porosity in different regions of the implant which are specifically designed to optimize ingrowth of different tissue and cells, to optimize the ability of the implant to withstand varying mechanical loads at specific regions of the implant, and to deliver growth agents to various portions of the implant in a controlled manner.

### RELATED APPLICATION

This application is a Continuation of U.S. patent application Ser. No. 09/957,829, filed Sep. 21, 2001, and entitled “Non-Uniform Porosity Tissue Implant,” which claims priority to Provisional Application Serial No. 60/234,841, filed Sep. 22, 2000, and entitled “Non-Uniform Porosity Tissue Implant, each of which is specifically incorporated herein by reference.

### BACKGROUND OF THE INVENTION

1. Field of the Invention

This invention relates to novel methods, for the production of tissue implants and prosthetics, including but not limited to orthopedic implants and prosthetics which have a controlled and directional gradient of porosity moving through all or one or more portions of the implant, as well as the implants produced by such a method.

2. Description of the State of Art

The advantage of porous materials, in general, is their ability to provide biologic fixation of the surrounding bony tissue via the ingrowth of mineralized tissue into the pore spaces. This is accomplished by increasing the available surface area for apposition (or bony contact) by having the interior of the implant accessible via pore spaces. Numerous factors may affect bone ingrowth into the pore spaces of these implants. Some of these factors include, but are not limited to, the porosity of the implant material (pore size, pore gradient, percent porosity), the time of implantation, material biocompatibility, depth of porosity into the implant, implant stiffness, amount of micromotion between the implant and adjacent bone.

The architecture of bone is such that the resulting porosities are non-uniform in nature. This is readily apparent in the longitudinal cross section of whole bone where the bone at the ends has the appearance of a sponge (cancellous or trabecular bone) while the bone at the center of the bone shaft is dense with little porosity (cortical bone). Nonuniform porosity is apparent in bone even at the microscopic level. At this level, vascular channels (Haversian and Volksmann canals) are approximately 100-250 microns diameter. Captured bone producing cells (lacunae, 5-10 microns diameter) and interconnecting fenestrations (canaliculi, 1-5 microns diameter) are examples of the low end of the porosities present in bone. Thus the range of porosity in normal bone is approximately from 1-5,000 microns.

It has been shown in numerous studies that the architecture of a porous implant has great effect on the ingrowth of bone into the pore spaces. For instance, evidence indicates that the optimum range of porosity for bone ingrowth is 100-400 microns. It has also been established that the pores must be interconnected in order to maintain the vascular system needed for continued bone development within the pore spaces along with increasing the initial fixation and fatigue strength of the implant. More recently, it has been shown that bone ingrowth into a porous implant placed in the maxilla (upper jaw) of humans decreases in a linear fashion as the depth into the implant increases. Bone ingrowth into the pores is 60% (that is, 60% of the available pore space is filled with bone) at the outer surface and decreases linearly, leveling off to approximately 15% bone ingrowth after 1000 microns depth. While this relationship is affected by time of implantation (shifting the line up or down) the piecewise linear relationship of bone ingrowth as a function of depth into the implant remains.

Time of implantation also indicates nonuniformity of bone ingrowth into porous implants. This is evidenced by the observation that at a given depth in the implant, bone ingrowth will asymptotically approach a maximum value over time. This value is affected by the location within the implant (e.g. at the surface or in the interior) with greater ingrowth values being obtained at the outer surfaces of the implant.

Porous implants and implant coatings approved for clinical use employ uniformly porous materials (e.g. mean pore size and percent porosity are uniform throughout the implant or coating). Depuy Porocoato®, Sulzer CSTi®, Interpore ProOsteono® are commercially available examples. Current implants with nonuniform porosities (e.g. porous nitinol) exhibit no directional gradient in porosity (i.e. vector). Nonuniform porosities may be present in the implant material but are placed randomly. Other implants may exhibit nonuniform porosities, as seen in the use of replaniform biomaterials (e.g. converted corals). There may exist a bimodal distribution of porosities, but no gradient from one pore size to the next is apparent in these natural implants.

There is still a need, therefore, for the manufacture of an implant that would more accurately mimic the architecture of natural bone. In so doing, this would encourage bone to grow into the pore spaces, providing a biological interlock between the implant and the surrounding bone. Such an implant would also better mimic the mechanical properties of whole bone further encouraging continued bone growth and maturation within the pore spaces.

### SUMMARY OF THE INVENTION

The invention described herein is a nonuniformly porous orthopedic implant. The implant may consist of a prosthesis with a nonuniformly porous outer surface or coating, or a finite number of layers with varying porosity with respect to each other, or said implant may be nonuniformly porous throughout its entire structure. The implant may be for use in any application in which porous orthopedic implants are indicated (e.g. hip/knee replacement, craniomaxillofacial reconstruction, etc.). Pore size diameter can be in a range from less than 5 μm to greater than 1,000 μm with transitions from one pore size to another occurring across the entire implant, or within successive sections via a porosity gradient.

Nonuniform porosity refers to a controlled gradient from a given pore size and/or percent porosity to another pore size and/or percent porosity that has a specified alignment or direction within the implant. The implant may contain a porosity gradient created by “stacking” lamina, each with differing uniform porosities. The porosity gradient may also be functionally graded such that the transition from one porosity to another is smooth (e.g. no step function) with no abrupt transitions. A functionally graded porosity may follow a linear transition between porosities. More complex functional gradients may be described logarithmically or exponentially (as 2 examples). Even more complex nonuniformly porous materials may be composed of functionally graded lamina “stacked” together. Porosities may be open (e.g. interconnected) or closed or some combination therein.

Additional objects, advantages, and novel features of this invention shall be set forth in part in the description and examples that follow, and in part will become apparent to those skilled in the art upon examination of the following or may be learned by the practice of the invention. The objects and the advantages of the invention may be realized and attained by means of the instrumentalities and in combinations particularly pointed out in the appended claims.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

In the Drawings:

FIG. 1 is a schematic representation of the dorsal view of a rabbit cranium showing approximate positioning of the nitinol implants.

FIG. 2 is a photomicrograph of a transverse cross-section of the parletal bone and an Implant Type #1 Bone ingrowth (B) Into the implant (I) can be seen throughout the cross-section. Cranial marrow cavity is denoted (MC), and internal surface of the parietal bone is denoted by (IS). (10× original magnification).

FIG. 3 is a photomicrograph depicting bone ingrowth into Implant Type #1. Apposition of ingrown bone can be seen in the pore spaces and at the interface of the implant. B: bone, I: implant. (25× original magnification).

FIG. 4 is a graphic representation of the microhardness of a viscoelastic Material.

FIG. 5 is a graphic representation of the microhardness of a viscoelastic Material.

FIG. 6 photomicrograph of a 4 month implant. Woven Bone (A) is forming in the porous HA block (B), with the majority of pore space occupied by vascular and soft tissue (C). ×25 magnification.

FIG. 7 photomicrograph of a 39 month implant. Lamellar bone (A) occupies a large portion of the available space. Some woven bone (B) is present. Porous HA block (C) and void space/soft tissue are also noted (D). ×25, magnification.

FIG. 8 photomicrograph of a 138 month implant. Only Lamellar bone (A) is present. Surrounding bone tissue (B) as well as porous HA block are noted (C). ×25 magnification.

FIG. 9 there were no significant differences between bone surrounding the implant, bone microhardness, as well as no significant differences in porous block hydroxylapatite microha bars denote one STD.

FIG. 10 is a graphic representation depicting the correlation of the number of Haversian systems per area of implant cross-section imaged to time of implantation, p<0.05.

FIG. 11 is a graphic representation depicting the correlation of the number of Haversian systems per area of implant imaged, normalized to the actual bone present within the implant (%I A-S), to the duration of implantation, p<0.05.

FIG. 12 is a schematic of implant sectioning and sequential imaging of the interfaces. Top diagram transverse cross-sections of the entire implant biopsy, Section A-A shows successive images taken to cross-section image.

FIG. 13 is a graphic representation of ingrowth over the depth of section into the implants.

FIG. 14 is a graphic representation of ingrowth as a function of implantation time at each incremental depth.

FIG. 15 is a graphic representation of the composite average of all data over time at all depths.

FIG. 16 is a graphic representation of the residuals of ingrowth as a function of implantation time compared to patient age.

FIG. 17 is a schematic representation of the relation between bone ingrowth, apposition and material.

FIG. 18 is a schematic representation of the relationship of bone ingrowth to apposition over time.

### DETAILED DESCRIPTION OF THE PREFERRED EMBODIMENT

The present invention is generally directed to a method for the production of tissue implants and prosthetics, including but not limited to orthopedic implants and prosthetics which have a controlled and directional gradient of porosity moving through all or one or more portions of the implant, as well as the implants produced by such a method. The non-uniform porosity gradient may be linear or more complex, and is preferably produced to have a continuous gradient within the desired regions. The desired effect is to create an implant which more closely mimics the natural structure of bone, and which improves the quality of the bone growth that occurs within the implant. In addition, implants can be created with varying porosity in different regions of the implant which are specifically designed to optimize ingrowth of different tissue and cells, to optimize the ability of the implant to withstand varying mechanical loads at specific regions of the implant, and to deliver growth agents to various portions of the implant in a controlled manner. The present inventor has defined the qualities of bone which can be mimicked using the method of the present invention, and this work is described in the Examples which follow. Since the production of the gradient can be controlled and altered to adapt to a particular environment or application (i.e., rather than as a random effect of the production process), the implants of the present invention provide great flexibility for use in a variety of implantation scenarios.

Preferably, the implant of the present invention is produced by modifying and/or adapting a known process of Self-propagating High Temperature Synthesis for use with a large variety of different materials, including the alloy, nitinol, to construct the graded porosity material. This method (and modified/adapted versions thereof), unlike any other known method for producing porous materials, allows for the use of any equation for the directed and controlled formation of any desired porosity combination and location in the implant. Other materials which can be used to form the implant include, but are not limited to, titanium, glass, ceramics, mixtures thereof and any other material which can be formed using a process such as Self-propagating High Temperature Synthesis. It is further noted that although Self-propagating High Temperature Synthesis, and modified or adapted methods thereof, are the preferred methods of creating the novel implants of the present invention, other methods known now or in the future which are capable of achieving the controlled, graded porosity implants of the present invention are intended to be encompassed by the present invention. The method of the present invention allows for the variation of the chemical constitution of the implant in a seamless, controlled manner; for the variation of the materials within the implant in a seamless, controlled manner, and for the variation of the porosity within the implant in a seamless, controlled manner.

As listed, the following are important advantages of this invention. Those who might find this invention useful include orthopedic device manufacturers, surgeons, and surgical patients undergoing orthopedic device implantation:

1) Nonuniform porosity to provide a framework for a nonuniform composite to be composed of reagent infiltration of the porosity (e.g. PMMA, bone cement, PGA/PLA, polymers, etc.).

2) Nonuniform porosity within sections to allow the use of differing infiltrating reagents (e.g. PMMA, bone, polymers, etc.) to form a “sandwich” implant.

3) Nonuniform porosity to mimic whole bone cross-section with porous sections simulating trabecular (cancellous) and cortical bone. (Trabecular (cancellous) bone refers to highly porous bone with pores (trabecula) ranging in size from less than 1 mm to upwards of 5 mm. Cortical bone refers to highly organized bone with little porosity other than that for vascular channels and entrapped cells.)

4) Functionally graded closed pore implant to create a prosthesis with specific localized mechanical properties, which can be held within the bone using other means (e.g. bone cement, screws, etc.), e.g. porous implant for craniofacial applications.

5) Nonuniform porosity to match tissue ingrowth to the material making up the implant or prosthesis (e.g. a laminate of porous HA and porous Ti).

6) Nonuniform porosity to match localized load conditions as experienced by the implant to sustain appropriate mechanical environments required for continued tissue development.

7) Device for the delivery of specific bone affecting reagents based upon reagent molecular weight. Reagents with high molecular weight will have a high viscosity/surface tension and therefore will not “flow” into smaller pores. Conversely low viscosity reagents will “flow” into the smaller pore spaces, thus allowing a functionally graded porous implant to have regions where specific reagents are located.

8) Nonuniform porosity specific to the bone growth mechanisms (e.g. intramembranous, endochondral, etc.) and specific to the bone into which the implant is placed (e.g. cranial, femur, pelvic, etc.). (Intramembranous bone growth refers to the direct development of osteoblasts and subsequent osseous tissue from the infiltrating mesenchymal tissue in the pore spaces. Endochondral bone growth refers to the differentiation of mesenchymal tissue within the pore spaces into chondrocytes prior to the deposition of bone.)

9) Nonuniform porosity to match the growth rate of bone ingrowth into a porous implant. Initial bone ingrowth within the pore spaces is at a much higher rate than older, remodeling bone. Bone ingrowth values begin to plateau at around 20 months post-implantation. Thus a higher percent porosity would be used in regions where initial bone ingrowth and maturation occurs (e.g. the surface of the implant) while lower percent porosity would be in regions where it has taken longer for bone to develop (e.g. in the implant interior). These regions would be interconnected via a functional gradient ensuring a continuum of bone and vascular soft tissue from one region to the next.

10) Nonuniform porosity for directed tissue ingrowth (e.g. pore size under 75 mm. to allow soft/vascular tissue access to specific prosthetic sites with larger pores (greater than 100 mm) allowing osseous tissue ingrowth at other locations).

11) Nonuniform porosity of sufficient size to allow for the mechanical interlock of pairing prosthesis (e.g. Mito® suture anchors, acetabular cup, etc.)

The presented implant design is not obvious because standard manufacturing processes used to create porous implants and implant coatings do not readily allow for the creation of variable porosities. The processes used to produce porous materials utilize uniformly sized beads or fibers sintered to, deposited or placed onto, a surface. In this manner a porous device is built up from a given surface. Due to the uniformity of the solid structures, the mean pore size and percent porosity of the material is uniform. Nonuniformities may exist but are randomly distributed and remain as a consequence of the, manufacturing system. A porosity gradient may be created using these standard processes. This is not done because the disadvantage of such processes are the increased complexity of manufacture, closed porosity, potential for alteration of the mechanical proper-ties of the underlying prosthesis and porous layers, distortion of the underlying porous layer (e.g. collapsing or realignment of the pore spaces) rendering the implant ineffective. Porous polymers and ceramics may be formed using a sol-gel process; however, in this technique a directed nonuniform porosity is difficult to obtain because of the use of a uniformly mixed colloidal or molecular solution.

Until recently, it had not been shown that bone ingrowth into porous biomaterials, is nonuniform both through the depth of the implant and over the time the implant is in vivo.

The invention is further illustrated by the following non-limited examples. All scientific and technical terms have the meanings as understood by one with ordinary skill in the art. The specific examples which follow illustrate the methods in which the non-uniform porosity implants of the present invention may be prepared and the non-uniform porosity implants themselves and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLES

The examples herein are meant to exemplify the various aspects of carrying out the invention and are not intended to limit the invention in any way.

### Example 1

One embodiment of the present invention is described below and is provided for illustration purposes and is not intended to limit the scope of the present invention.

The utility of nitinol as a superelastic, shape-memory alloy implant material has yet to be fully investigated. Nitinol, or porous, equiatomic NiTi shape memory alloy (approximately equal atomic masses of nickel and titanium), has recently been investigated as a material for craniofacial applications (Simske and Sachdeva 1995; Ayers et al. 1999). In Russia, China and Germany, it has been in clinical use for approximately a decade in maxillofacial surgeries and other orthopedic procedures involving thousands of patients (Shabalovskaya 1996; Dai 1996; Airoldi and Riva 1996). Porous nitinol can be produced by various manufacturing processes, including, but not limited to, sintering of molten NiTi and self-propagating-high-temperature-synthesis (SHS) (Itin et al. 1994; Yi and Moore 1990). Such methods allow for a controlled range of NiTi porosity, and provide appropriately sized and interconnected (open) pores, creating an implant morphology similar to bone. A porous implant structure allows ingrowth of mineralized tissue, establishing a biological fixation of the implant. It has been shown that 50% porous NiTi provides greater initial bone ingrowth (as a percentage of the implant cross-section) than 30% porous hydroxyapatite, primarily due to the greater exposed surface area (Simske and Sachdeva 1995). Moreover, NiTi in this porosity range provides a void space, after bone ingrowth, similar in percentage of cross-section to that of rabbit cranial bone further indicating NiTi's ability to at least architecturally mimic bone (Simske and Sachdeva 1995). The shape memory property of NiTi also allows for the possibility of in situ implant shape in the case of injury to the implant or surrounding hard tissue.

The superelasticity and high strength material properties of nitinol also suggest its candidacy for orthopedic implantation. The superelastic properties allow the surgeon greater margin in sizing bony defects as the implant can be press-fitted into the bone without unduly damaging the surrounding bone or implant. In fact, such a press fitted superelastic shape-memory alloy may naturally space surrounding bone through cyclic resorption. The high strength of NiTi (UTS of 895 MPa, annealed) allows for good initial fixation of the implant by withstanding the stresses induced by mastication or other imposed loads. With the incorporation of porosities into the NiTi, the potential for the matching of the mechanical properties of the implant to the surrounding bone becomes available, decreasing the prevalence and magnitude of subsequent stress-shielding.

Metals and ceramics in current clinical use have a modulus of elasticity in the range of 100-400 GPa. This is in contrast to bone, which has an elastic modulus an order of magnitude less (20 GPa for cortical bone with approximately ⅔ mineral mass percentage of dry mass). The martensitic modulus of elasticity for solid NiTi is in the 28-41 GPa range (close to the modulus of bone). By making NiTi 50% porous, the apparent modulus of the implant is below the range of bone (14-20 GPa). If an exact match between a bone infiltrated implant and the surrounding bone is required to minimize stress-shielding, the low modulus of porous NiTi allows the possibility of significant ingrowth at this matching value. Itin et al. demonstrated further the ability of NiTi to mimic the mechanical properties showing 40-50% porous nitinol has a recoverable strain of 3.2% near physiologic temperatures, which is similar to the recoverable strain of bone at 2% (Itin et al. 1994). This important aspect of NiTi superelasticity suggests that if the surrounding bone is strained within its elastic region (less than 2%), the implant will deform with the bone and recover its original shape afterwards, preserving the implant/bone bond.

This review examines the most common types of porous biomaterials in clinical use for craniofacial applications, developing a hypothesis about what constitutes an effective porous orthopedic biomaterial. Next, it discusses the biocompatibility of NiTi. This, in turn, springboards a discussion about the advantages and disadvantages of NiTi as a porous biomaterial by comparing NiTi to commonly used orthopedic biomaterials. Future work necessary to characterize porous NiTi as a material for bone engineering is then presented.

Porous Biomaterials in Craniomaxillofacial Applications

The advantage of porous materials, in general, is their ability to provide biologic fixation of the surrounding bony tissue via the ingrowth of mineralized tissue into the pore spaces. This is accomplished by increasing the available surface area for apposition by having the interior of the implant accessible via pore spaces (Greene et al. 1997). It has been established that mineralized tissue ingrowth requires pore sizes in the range of 100-400 microns (Klawitter and Hulbert 197 1; Hulbert et al. 1970). Such morphology allows for early rapid cartilaginous ingrowth and subsequent bone maturation over the lifetime of the implant. An open porosity (interconnected pores) allows for vascularization to support osseous tissue ingrowth and continued bone maturation (vanEeden and Ripamonti 1993). This architecture is analogous to the perpendicular aspects of bone morphology, exhibited at the vascular level by Haversian and Volkmann's canals. Interconnected pores increase stability and cosmesis of the bone (Kent and Zide 1984; Wolford et al. 1987) and increase resistance to fatigue loading (Epply and Sadove 1990). The increased stability (defined for the puposes of this paper as micromotion under 150 μm (Bragdon et al. 1996; Ramamurti et al. 1997)) reduces implant micromotion and the resultant resorption of adjacent bone (Kent and Zide 1984) or inhibition of cartilaginous ingrowth (Bragdon et al. 1996).

Porous materials likely affect bone ingrowth into the implant pores by matching the mechanical properties of the interface to the surrounding bone, reducing stress-shielding through a graded transfer of the stresses which are imparted at the implant/bone interface (Ayers et al. in press; Pedersen et al. 1991; Hollister et al. 1993). As such, one can enhance the efficiency of the load transfer between the implant and surrounding bone by optimizing the porosity (in terms of pore size, gradient and percent) of the implant to the bone into which it is placed and the loading environment to which it is exposed. Recent experiments indicate that pore spaces -also allow- the delivery of appropriate healing and growth factors to the ingrowing tissue. Thus, porous materials allow one to address both biologic and mechanical aspects imposed upon orthopedic implants during the initial phases of mineralized tissue ingrowth and its continued maturation.

In general, the predominant implant materials clinically used in oralmaxillofacial and craniofacial applications are autogenous bone, bank bone (such as antigen extracted autolyzed bone) and porous block hydroxyapatite (Interpore 200® is a commercial example of such a material in clinical use). Autogenous bone is the most common porous material used in craniofacial reconstruction (Phillips et al. 1992). The use of this material has the significant advantage of reduced rejection by the patient. Donor sites for autogenous bone include the rib, crania and iliac crest (Szachowicz 1995). Difficulties arise in the need for a secondary surgical site along with subsequent increases in operation time and the potential for donor site complications including, but not limited to infection, fracture and reduced patient ambulation (Kent and Zide 1984; Desilets et al. 1990; Motoki and Mulliken 1990). Bank bone may be used to eliminate the need for a second surgical site, but there still remains the disadvantage of improper bonding between the host bone and the graft and the potential for infection (Kent and Zide 1984). Microhardness data indicates oven-ashed bone may provide an alternative (Broz et al. 1996). Nevertheless, the resorption rates of autogenous and allogenic, bone grafts are unpredictable leading to the possibility of implant instability and implant failure (Kent and Zide 1984; -Szachowicz 1995; Phillips et al. 1992). A graft should be resorbed in such a manner that it allows sufficient time and structure for vascularization of the porosities and subsequent bone ingrowth (Phillips et al. 1992).

Slow resorption is a reason that ceramic biomaterials based on calcium phosphates (the mineral phase of bone) have gained favor. These materials include hydroxyapatite (HA) and tricalcium phosphate (β-TCP). They can be manufactured to provide for controlled resorption with appropriate porosity (Eggli et al. 1987; Kent and Zide 1984; Light and Kanat 1991). These ceramics have the disadvantage of being brittle and difficult to machine, but are strong enough to withstand the forces induced during mastication (Wolford et al. 1987; Holmes et al. 1988; Nunes et al. 1997). Dense hydroxyapatite in the form of porous block coralline HA is an effective material for use in craniofacial applications (Ayers et al. 1998; Nunes et al. 1997; Wolford et al. 1987; Holmes et al. 1988; Jahn 1992). It is also used as a porous coating for otherwise nonporous materials such as titanium, providing a large area for micromechanical fixation via osseointegration of the implant, increasing its stability during the early phases of bone ingrowth (Engh and Bugbee 1998; Ducheyne 1998).

In maxillofacial applications in humans, woven bone invades the porous HA in as early as 4 months up to 300 μm deep (Ayers et al. in press, Nunes et al. 1997). This early woven bone is then remodeled into lamellar bone and, subsequently, Haversian type bone (Ayers et al. 1998, Wolford et al. 1987; Nunes et al. 1997). Bone ingrowth progresses until about 20 months reaching an asymptotic condition at all depths in the implant, with the relative amount of osseous tissue remaining constant (Ayers et al. in press; Nunes et al. 1997). During this progression, the bone matures into Haversian-based bone, exhibiting its normal structural properties and metabolism (Ayers et al. 1998). The HA, meanwhile, may undergo modest resorption (Nunes et al. 1997; Martin et al. 1993).

The ideal implant for a variety of applications may have pore sizes that allow for rapid bone ingrowth and apposition with a porosity that matches the mechanical properties of the implant to the surrounding bone. This implant would also need to be bioinert, or preferably bioactive (osteoinductive and/or osteoconductive), and be resorbed over time at a rate that ensures stability and cosmesis of the surrounding bony structures. While porous NiTi is not resorbable, as the following discussion will highlight, it can be formed and treated to meet the other traits herein considered desirable in an orthopedic implant.

NiTi Biocompatibility

Numerous studies have examined the biocompatibility of NiTi in vitro and in vivo, with differing results. Rondelli, using human body simulating, fluids reported that NiTi has a localized corrosion resistance similar to Ti6Al4V, but when the passivation layer is abruptly damaged, NiTi's corrosion resistance is less than Ti6Al4V while is still being comparable to other austenitic steels (such as ASTM 316L) (Rondelli 1996). Putters et al., using the inhibition of mitosis in human fibroblasts cultured on nitinol, titanium and nickel substrates, stated that the results indicate that nitinol is comparable to titanium in its biocompatibility (Putters et al. 1992). Sarkar et al. showed that NiTi had an earlier breakdown of its passive oxide layer than other implant materials such as titanium, stainless steel and cobalt-chrome alloys when subjected to potentiodynamic, cyclic polarization tests in a sodium chloride solution (Sarkar et al. 1983). It should be noted, these studies focused on the surfaces of solid NiTi, thus, it may be expected that porous NiTi may have diminished corrosion resistance by the fact of its greater surface area in contact with bodily fluids.

In vivo work is generally supportive of NiTi's biocompatibility. Simske and Sachdeva, and more recently Ayers et al. have demonstrated that bone ingrowth into porous nitinol in the crania of rabbits is evident as early as six weeks and that bone contact is made with the surrounding cranial hard tissue (Simske and Sachdeva 1995; Ayers et al. 1999). A study using high purity nitinol alloy implanted in the femurs of beagles for 3, 6, 12, and 17 months showed no evidence of localized, or general corrosion on the surfaces of the implants and no metallic contamination of organs due to the implants (Castleman et al. 1976). Using quantitative histomorphometry, nitinol was shown to be progressively encapsulated by bony tissue in the tibiae of rats, albeit at a reduced rate when compared to pure titanium, anodic oxidized Ti and Ti6Al4V, over the course of a 168-day experimental period (Takeshita et al. 1997). In a finding similar to Takeshita et al., Berger-Gorbet et al., using immunohistochemistry, showed NiTi screws implanted in rabbit tibia had slower osteogenesis with no close contact between implant and bone as compared to screws made of c. p. titanium, Vitallium, Duplex austenitic-ferritic stainless steel (SAF), and 316L Stainless Steel (Berger-Gorbet et al. 1996). Clinical results of procedures using NiTi alloys in China and Russia state no significant detrimental effects of devices implanted in craniofacial bone (Shabalovskaya 1996; Dai 1996). However, the specific studies upon which this conclusion is made are not readily obtainable, making replication difficult.

Mechanisms of NiTi Biocompatibility

The biocompatibility of NiTi derives from the formation of an oxide layer (TiO₂) on the surface of the implant. This is similar to the TiO₂ layer formed on pure titanium, which enhances its biocompatibility as an implant material (Trepanier et al. 1998). The passivation layer can range in thickness from 2 nm-1 micron (Endo 1995; Trepanier et al. 1996). Resistance of this layer to damage correlates with the corrosion resistance, and hence biocompatibility, of the implant. Overall thickness of the passivation layer is less germane to biocompatibility than its uniformity (Trepanier et al. 1996). Because the oxide layer is a brittle ceramic, the superelasticity of the NiTi substrate can induce stresses in the passivation layer as the implant deforms causing cracking and resulting in a pitting attack of the NiTi substrate (Villermaux et al. 1996). Maintaining the integrity of, the passivation layer is paramount with nitinol to prevent the potential release of metallic nickel into the body. It has been established in the literature that nickel in vivo is highly toxic, producing severe inflammatory responses, along with being a potential carcinogen.

In order to preserve the substrate from pitting corrosion numerous methods of manufacturing the oxide layer have been examined. The easiest method is simple aging of the material in air, allowing for a natural oxidation layer to form. An associated side-effect, however, is that the oxide layer may contain metallic Ni and nickel-oxides at the NiTi surface (Trepanier et al. 1996; Shabalovskaya 1996). Steam or water autoclaving has been shown to reduce the presence of Ni, depleting it to a depth upwards of 10 nm into the NiTi substrate (Shabalovskaya 1996). The resulting oxide layer contains primarily TiO₂ based oxides (Shabalovskaya 1996). Heat treating the surface of NiTi in a nitrite/nitrate salt has been used to create a very thick oxide layer (approximately 0.1 microns), as compared to other treatments (Trepanier et al. 1996). However, this layer has been shown to contain a Ni rich region above the NiTi substrate, which could, if the oxide layer is damaged, result in dissolution of Ni from the implant (Trepanier et al. 1996). Heat treating also carries the risk of altering the mechanical properties of the NiTi. Two methods that produce thin but very uniform oxidation layers are passivation of the NiTi surface with nitric acid solution and electropolishing (Tepanier et al. 1996). Electropolishing significantly increases the corrosion resistance of NiTi (Trepanier et al. 1996).

Other methods for enhancing the corrosion resistance of NiTi involve the deposition of a non-metallic layer on the NiTi surface. This allows for the creation of thick (upwards of 1 mm) films on the NiTi substrate. One method that has shown promise is the plasma deposition of polymerized tetrafluoroethylene (PPTFE) (Villermaux et al. 1996; Yahia et al. 1997). This method approximately doubled the passivation range of NiTi in physiological Hank's solution and decreased the pit diameter by an order of magnitude when used on osteosynthesis staples (Villermaux et al. 1996). This passivation layer was also elastic enough to follow the large deformations induced by NiTi's shape memory effect without cracking (Villermaux et al. 1996).

Perhaps the most unique method of inhibiting the dissolution of Ni from the NiTi substrate involves creating a bioactive film. By creating a covalently bonded coupling layer between the Ti-oxide and immobilized human fibronectin, Endo was able to demonstrate increased corrosion resistance of the NiTi, along with the ability of the attached layer to withstand hydrolysis in solution at pH 4.0-7.0 (Endo 1995). This offers a unique opportunity for bone engineering in which a material that may be considered to neither support or degrade bone ingrowth (an osteopermissive material) (Ayers et al. 1999) can be made to be bioactive (similar to calcium phosphates such as HA). More importantly, this is a key extracellular matrix (ECM) compound upon which osteogenic cells attach and develop. Regardless, in the case of porous NiTi, whatever method is used to enhance the biocompatibility of NiTi it must be able to penetrate the interior pores of the material to ensure treatment of all of the implant's surfaces. The authors' have used steam autoclaving for 30 minutes. While the surface properties of the steam-autoclaved implants have not been analyzed, the implants prepared in this manner do allow for bone ingrowth and direct bone and implant contact (apposition).

Inventors' Experience with NiTi

The inventors' experiments have shown that porous nitinol is generally biocompatible when placed in the crania of rabbits, and deserves further study as a material for bone engineering. Studies conducted have examined the effects of NiTi porosity on rabbit cranial bone ingrowth at 6 weeks (Ayers et al. 1999) and bone ingrowth over a 12 week period with indirect comparison to the well-characterized cranial implant material HA (in the form of Interpore 200®) (Simske and Sachdeva 1995). In both of these studies, porous NiTi implants were placed in the parietal bone of New Zealand White rabbits in defects machined to the specific geometry of the implant. In neither experiment were macrophage cells noted adjacent to, or within, the implants. Soft and connective tissues readily adhered to the implants post-surgically. Both studies used uncoated (other than the oxide layer induced during autoclaving) porous equiatomic nickel-titanium (nitinol) implants.

The study examining the effect of porosity on bone ingrowth after 6-weeks; addresses two aspects of the use of nitinol in cranial bone defect repair. The first is the verification of substantial bone ingrowth into the implant after six-weeks. The second is the determination of the effect of pore size on the ability of bone to grow into the implant during the early (6-week) post-operative period. Implant specimens with three different morphologies (differing in pore size and percent porosity) were implanted for 6 weeks.

A quick synopsis of the data (Table I) shows mean pore size (MPS) of Implant Type #1 (353+/−741 μm) differed considerably from that of Implant Type #2 (218+/−28 μm) and Implant-Type #3.(178+/−31 μm).

TABLE I
Porous Nitinol Implant Morpholgy

| Measurement | Implant #1 (n = 7) | Implant #2 (n = 6) | Implant #3 (n = 7) |
|---|---|---|---|
| Thickness (μm) | 644 +/− 21* | 345 +/− 37 | 385 +/− 56 |
| % Volume Pore Space (Porosity) | 42.9 +/− 4.0* | 54.4 +/− 5.3 | 50.5 +/− 13.7 |
| Mean Pore Size (μm) | 353 +/− 74* | 218 +/− 28 | 179 +/− 31 |
| Available Pore Volume for | 6.9 +/− 0.6* | 4.7 +/− 0.7 | 5.1 +/− 2.0 |
| Ingrowth (mm³) | | | |

*Denotes measurements statistically significantly (P < 0.05, Tukey-Kramer HSD) different in Implant #1 when compared to either Implant #2 or Implant #3.

Quantitative histomorphometric measurements are presented in Table II, below.

TABLE II

| | Implant #1 | Implant #3 | Implant #3 |
|---|---|---|---|
| Measurement | (n = 7) | (n = 6) | (n = 7) |
| Percent Implant (%) | 57.1 +/− 4.0 | 45.6 +/− 5.3 | 49.5 +/− 13.7 |
| Percent Void (%) | 26.9 +/− 3.8 | 33.6 +/− 5.1 | 35.1 +/− 10.9 |
| Percent Bone (%) | 14.6 +/− 5.9 | 20.8 +/− 6.7 | 15.4 +/− 4.7 |
| Percent Ingrowth (%) | 37.4 +/− 7.8 | 37.9 +/− 10.1 | 31.1 +/− 6.9 |
| Bony Appostion, | 47.4 +/− 9/6# | 41.6 +/− 9.2 | 32.0 +/− 9.1 |
| Exterior (%) | | | |
| Bony Appostion, | 38.6 +/− 12.7 | 41.9 +/− 10.5 | 36.0 +/− 11.1 |
| Interior (%) | | | |
| Total Bone Ingrowth | 2.6 +/− 0.6# | 1.8 +/− 0.5 | 1.5 +/− 0.7 |
| (mm³) | | | |

Values are given as mean +/− standard error of the mean for each of the three implant types. An asterisk (*) indicates a significant difference (P < 0.05) from Implant #2- A pound sign#indicates a significant difference (P < 0.05) from Implant #3.

There were no significant differences between implant types in the percentages of bone and void/soft tissue composition of the aggregate implants. The amount of bone ingrowth was also not significantly different between implant types. Implant #1 was significantly higher in pore volume and thus had a significantly higher volume of ingrown bone (2.6+/−0.6 mm³) than Implant #3 (1.5 0.7 mm³); and a greater amount, but without statistical significance, than Implant #2 (1.8+/−0.5 mm³). The difference between implant types in total volume of bone ingrowth is ostensibly a function of the implant volume. Implant #1 had a greater volume available for bone ingrowth. The difference in Implant #I1's external bony apposition most likely reflects the greater surface area for bony contact of Implant #1 as compared to the other implants.

In thin implants (i.e. implant thickness is on the same order of magnitude as pore size) pore size does not appear to affect the bone ingrowth during the cartilaginous (analogous to fracture repair) period of bone growth within the implant. This implies that over the commonly accepted range of implant porosities (100-400 μm), the bone ingrowth near the interface of nitinol implants at six weeks is similar. Surface contact (apposition) measurements were also used as gauge of the biocompatibility of the implants as this is an accepted general measure of biocompatibility (Simske and Sachdeva 1995; Ono et al. 1990). The measurements (Table II) do not imply that nitinol is osteoconductive, but indicate that it does not inhibit bone ingrowth in the early healing phase of the defect.

In another study (Simske and Sachdeva 1995), geometrically equivalent (5×5×1 mm.) uncoated porous nitinol and coralline hydroxyapatite (HA, Interpore 200®) implants were placed 4 mm to either side of the midsection of the frontal bone and 4 mm anterior to the coronal suture of the cranial bone of New Zealand White rabbits. The rabbits were killed at each of three postsurgical intervals (2,6 and 12 weeks), and the implants were evaluated for gross biocompatibility, bony contact and ingrowth.

Histologically, bony contact was present for both materials. Both materials made bone contact with the surrounding cranial hard tissue, and percent ingrowth increased with surgical recovery time. Measurements of microhardness in conjunction with bone histological observations indicate that bone within and in contact with the implants is similar in site-specific structural proper-ties to the surrounding cranial bone. Porous nitinol implants appear to permit significant cranial bone ingrowth after as little as 12 weeks, and thus nitinol appears to be suitable for craniofacial applications. Compared to HA, the nitinol implants demonstrated a trend for less total apposition and more total ingrowth after 6 and 12 weeks of implantation (Table III).

TABLE III
Quantitative Histomorphometry for Porous Nitinol and Hydroxyapatite

| Implantation | Implant Apposition (%) | | Implant Ingrowth (%) | |
|---|---|---|---|---|
| Time | HA | NiTi | HA | NiTi |
| 2 weeks (n = 2) | 12.5 ± 12.5 | 9.2 ± 9.2 | 0.0 ± 0.0 | 0.0 ± 0.0 |
| 6 weeks (n = 2) | 39.0 ± 4.8 | 34.9 ± 0.5 | 6.7 ± 6.7 | 12.2 ± 0.5 |
| 12 weeks (n = 3) | 50.4 ± 4.2 | 39.6 ± 6.6 | 25.3 ± 9.3 | 34.3 ± 11.4 |

These results may be due to the osteoconductive properties of HA (Neo et al. 1998, Ono et al. 1990) or to the differences in the surface morphologies between the implants used in this study. The nitinol, with a greater surface porosity (50%) than the HA (30%), may have allowed readier access to the interior of the implant than the HA.

NiTi vs. Other Biomaterials

Mechanical Considerations

One of the primary concerns of bone engineering arises from the premise of “Wolff's Law”: that bone not subjected to loading undergoes resorption. When an implant with an elastic modulus stiffer than bone is used, mechanical disuse causes the surrounding, bone to resorb (stress-shielding), threatening the stability of the implant. Thus, matching the material properties of the implant to the bone for a given application may be paramount to the success of a porous metal implant. Material property matching is perhaps less important in craniofacial applications than in joint replacement (e.g. hip and knee arthroplasty), due to the different mechanisms governing bone growth (Rawlinson et al. 1995). Nonetheless, the mechanical aspect of craniofacial implantation must be considered (Ayers et al. in press).

It would be inappropriate to assign a single value to the elastic modulus of solid NiTi because the elastic modulus is nonlinear with respect to temperature. The martensitic elastic modulus follows the Clausius-Clapeyron equation in the form of ∂σₐ/σMₛ=ΔH/Tε₀ where σₐ is the applied stress, Mₛ, is martensitic temperature, so ε₀ is the transformation strain resolved along the line of the applied stress, ΔH is the transformation latent heat and T is the temperature (Otsuka and Wayman 1998). Thus, there is a family of stress-strain curves dependent upon temperature for a given specimen. When porous, determining the structural modulus of the implant is further complicated. For example, at a temperature of 293° K, the modulus of 40-50% porous nitinol is approximately 25 GPa (Itin et al. 1994). This compares to standard biomedical titanium alloys such as solid Ti6Al4V with a modulus of 1 10 GPa. Other metals such as ASTM 316L and CoCr alloys have elastic moduli of 200 and 220 GPa, respectively if they are solid. Roughly, the metals used in clinical applications are an order of magnitude stiffer than bone, while 40-50% porous NiTi is similar to bone in stiffness.

Formation Considerations

Metals such as Ti6A14V and CoCr are not normally manufactured in a porous form. They can be made “porous”, however, by coating the outer surfaces with metal powders via plasma spraying either metal or ceramic powders onto the metal surface; or by double sintering metallic beads onto the heated metal substrate. Pore sizes can range from 150-300 microns using these techniques with percent porosity from 20-40%. While porous coatings may enhance the osseointegration of the implant, it has been shown that the bond between bone and coating is preserved better than the bond between the coating and the substrate, resulting in the possible failure at the implant coating/substrate interface (Spector 1987; Vercaigne et al. 1998).

Ceramics occur naturally as porous materials (e.g. bone, coral, etc.) or can be manufactured to be porous via numerous methods including combustion synthesis, sintering, and plasma spraying. There are at least nine recognizable biodegradable bioceramics that are used in bone engineering (Bajpai and Billotte 1995). These are aluminum-calcium-phosphorous-oxides, glass fibers and their composites, corals, calcium sulfates, ferric-calcium-phosphorous oxides, hydroxyapatite, tricalcium. phosphate, zinc-calcium-phosphorous oxides and zinc-calcium-phosphorous oxides. In addition, Bajpai and Billotte list six bioinert ceramics including pyrolitic carbon coated devices, dense hydroxyapatites, dense nonporous aluminum oxides, porous aluminum oxides, zirconia and calcium aluminates. Surface reactive bioceramics include bioglasses and ceravital, dense and nonporous glasses and hydroxyapatite (Bajpai and Billotte 1995).

The elastic modulus of the bioceramics mentioned above range from 40-117 GPa for pure crystalline hydroxyapatite to as high as 400 GPa for corundum. These values can also be adjusted based upon the natural or manufactured porosity of the materials. For example, the elastic modulus of corals, which are predominately hydroxyapatite, changes by an order of magnitude over a porosity range of 0-5 0%; thus, a 100 GPa modulus can be reduced to 10 GPa in a highly porous form (3 0-50%). The apparent modulus of the porous forms of porous materials may be estimated via the equation E=Eₛ,(Vₛ,^{x} where E is the apparent modulus, Eₛ is the elastic modulus of the solid; Vₛ is the volume fraction of the of the solid phase; X is a variable ranging from 1 to 2, being approximately 1 when Vₛ is approximately 1 and approximately 2 when Vₛ is approximately 0 (Lakes 1995). Given this, it is apparent that within an acceptable range of porosities, ceramic and glass materials can be manufactured to have apparent densities that of bone.

Machining

Machining considerations must also be taken into account when comparing these materials. This consideration arises from the need for the surgeon to be able to match the implant to the bony defect during the surgery to provide the best possible match between the implant and surrounding bone. Ceramics are very brittle, and are difficult to machine: warnings about the brittleness are prevalent in the literature. This is largely mitigated by the ability to form the ceramic into the appropriate shape beforehand, reducing the need for post-production machining. Porous metals formed by sintering or the plasma spraying of powders and diffusion bonding of metal fibers to a metal substrate can result in the damage to the underlying substrate and a coating that is also brittle and difficult to machine (Simske et al. 1997). Self-propagating-high-temperature-synthesis (SHS) has, nevertheless, allowed the manufacture relatively complex shapes in nitinol (cones, polygons, etc.) reducing the need for post-production machining. The use of SHS in the formation of nitinol allows implants to be created very rapidly (on the order of seconds to minutes) in contrast to sintering or diffusion bonding processes, which can take hours to days to complete (Yi and Moore 1990).

Biocompatibility

Ceramics and glasses such as HA, TCP and bioglasses are quite biocompatible. They promote the differentiation of the osteoblast phenotype from marrow stem cells, and are thus, osteoconductive in addition to being biocompatible. Another advantage of these ceramics over metals such as nitinol is their ability to degrade over time, allowing bone to, fill in the implant space. While the biocompatibility of NiTi is still under study, it has been our experience that NiTi is bioinert in vivo. It acts as an osteopermissive (or bioinert, similar to pure Ti and its alloys) material simply providing a scaffold upon which the bone may grow, neither promoting bone formation nor preventing it. As has been discussed earlier, the passive oxide layer can render NiTi bioactive similar to HA, TCP and bioglass. There does exist sufficient clinical evidence that over long-term implantation NiTi remains inert while metals such as ASTM316L Stainless Steel, which have been optimized for corrosion resistance (hence biocompatibility), will corrode.

Porous NiTi formed and machined into an implant mimics the mechanical and material proper-ties of bone. It is sufficiently ductile to be machined in an operating theater without the need for specialized equipment or processing. While it is not bioactive like many of the ceramics, there is the potential to make it so (via coatings, impregnating reagents, etc.).—Perhaps the greatest draw back is that NiTi is not biodegradable. This can be an advantage, however, when repairing large defects caused by congenital bone diseases or non-union fractures wherein the normal mechanisms for bone growth are no longer present.

Present and Future Advantages of Porous NiTi

The advantages of NiTi over current implant materials are in its superelasticity at body temperature, ease of formation and versatility in creating graded open porosities. With a forming process such as SHS, one can readily create a wide variety of pore size and porosity combinations in almost any shape. While SHS can be used to create porous Ti, Ti alloys and other metals, NiTi again has the advantage of being a superelastic shape memory alloy. These properties allow the surgeon greater leeway in implant placement and better chance of saving the implant in the case of traumatic injury (i.e. fracture) in the area the implant is located (in situ implant shape recovery).

Porous NiTi's superelasticity is maintained even after bone ingrowth satisfying the need for biomechanical compatibility (Itin et al. 1994). This advantage of NiTi over other implant materials opens several avenues of orthopedic treatment heretofore unavailable. The ability of 40-50% porous NiTi to undergo upwards of 3.2% recoverable strain means an implant is more likely to remain integrated with the bone when subjected to peak physiological stresses such as those noted during a stumble when climbing stairs (870% body mass) (Bergmann et al. 1995) which may deform the bone beyond the elastic deformation limits of implant materials in current use (note that 3.2% is even greater than bone's own recoverable strain of approximately 2%). Superelasticity may also be used in limb elongation procedures. To accomplish this the implant is preloaded prior to implantation. Upon its osseointegration, thermoelectrical stimulus can be used to return the implant to its original shape. NiTi allows this to be done in small incremental steps with constant stress on the surrounding bone, reducing patient discomfort. A similar method is used in orthodontic archwires in humans (Airoldi and Riva 1996) and in scoliosis correction in goats (Schmerling et al. 1976).

Other advantages of NiTi as a porous biomaterial arise from its ability to be produced via SHS. This method of formation relies on the exothermic reaction of nickel and titanium powders when heated to their combustion temperature of 1773° K (Yi and Moore 1990). When a gassifying reagent such as B₂O₃ is added, porosities are created. The pore size and porosity can be controlled based upon the amount of gassifying agent, pressure of the reaction chamber and/or gravitational forces. This process allows the creation of complex shapes (r educing the amount of secondary processing and machining) in very short time periods (order of minutes). Perhaps, in the future, the patient will undergo a CT scan at the specific site in need of repair, and a mold may be created using stereolithogrophy or a similar technology. This mold would be filled with the appropriate mixture of nickel, titanium and gassifying agents and ignited, creating a custom implant for the specific patient application in a matter of a few days.

Much work has yet to be done to fully characterize porous NiTi as a material for bone engineering. This work ranges from refining the formation and processing of NiTi to rendering NiTi bioactive. In the area of materials processing, it has been demonstrated that ceramics can be combined with NiTi to create a composite or aggregate material (Itin et al. 1997). The incorporation of a superelastic shape-memory alloy enhances the tensile strength properties of the ceramic, while the ceramic provides the bioactivity for increased ingrowth of tissue (Itin et al. 1997). It is very feasible that a NiTi core with a bioactive ceramic outer surface can be created using SHS. There would be no interface between the ceramic and NiTi, as the transition from one to the other would occur over a functional gradient. In so doing the material and mechanical properties of the surrounding bone are matched with the ceramic, providing a bioactive surface for osseointegration, reducing the time for mineralized tissue infiltration and consequently patient recovery time.

SHS production of NiTi allows one to quantify the nature of bone ingrowth into porous NiTi. In craniofacial applications, it has been proposed that in an approximately 65% porous block coralline HA implant with a mean pore size of 230 μm the mechanical transfer of loads occurs within the first millimeter of the implant surface (Ayers et al. in press). If this is the case, are interior porosities needed? These questions may be answered by creating implants with functionally graded porosities, where the surface pore size is sufficient to allow for a rapid influx of tissue and scales down towards the center of the implant. Depending on the implant application, the interior could remain solid for implants subjected to high loading environments, or be porous, allowing for vascular tissue ingrowth and later bone maturation.

As has been discussed, NiTi offers the advantage of the implant being matched to the mechanical properties of the bone. On the other hand, NiTi is not considered to be as biologically advantageous as other implant materials; for example hydroxyapatie. However, Cytokine infiltration of NiTi pore spaces and/or bio-coating the NiTi surface may bridge this gap between NiTi's osteopermissive nature and HA's osteoconductivity. Cytokine infiltration of implants is the addition of bone affecting proteins into the pore spaces of the implant. This offers the opportunity to improve the initial fixation at the bone/implant interface by enhancing the early development of osseous tissue. To highlight this case, biodegradable porous implants are beginning to be used as devices for the delivery of bone affecting proteins (Schwartz et. al. 1998; Gao et al. 1997; Guicheux et al. 1998). Porous NiTi infiltrated with bone affecting proteins could utilize a similar principal with a specific local response as the goal; given that reagents appropriate for the time course of bone growth in the implant are considered (Hollinger 1993). Of course, NiTi is not biodegradable, thus its permanence at the repair site would need to be taken into consideration.

Infiltration into the implant pore spaces can use any bone-affecting reagent. The mechanisms for bone formation or inhibition of resorption would be possible target pathways. In other cases, controlled resorption in one area and formation at another may be desired. As such, release kinetics must be considered when choosing a target. An examination of bone morphogenic protein (BMP) release in microporous polylactic/polyglycolic acid (PLA-PGA) implants was examined in physiologic PBS for 72 days (Agrawal et al., 1995). An initial BMP “burst” was released in the first four days. BMP continued to desorb from he PLA-PGA beyond two months at levels approximately an order of magnitude less than the initial burst. One may expect a similar temporal response in nitinol surface treated with the same BMP. With this anabolic bone proteins may be better candidate reagents to consider than anti-resorptives. An anti-resorptive would serve to prevent bone turnover at the interface between bone and implant or to prevent a stress shielding response. The above study (Agrawal et al., 1995) suggests that the kinetics of protein release would not be appropriate for preventing the longer-term resorption. However, long-term resorption should be mitigated by the very nature of the permanence of the NiTi implant and its structural/mechanical mimicry of mature bone.

BMP infiltration of implants is the most common protein currently being examined to promote growth of bone. Bone formation has been initiated using Plaster of Paris (PLOP) infiltrated with bovine BMP improving the healing of human femoral non-union fractures in patients who had undergone unsuccessful surgeries to repair the defects (Meng-Hai et al., 1996). Human demineralized bone allografts infiltrated with BMP-2 promote bone ingrowth into otherwise inactive implants (Schwartz et al., 1998). BMP in a coral implant has been examined in the repair of a tibial defect in sheep (Gao et al., 1997). Significantly increased bone ingrowth was noted in the firs six weeks, as compared to coral controls. After 16 weeks of implantation mechanical testing showed a trend towards decreased mechanical properties of the BMP impregnated implants as compared to controls. This was explained by the presence of high concentrations of anti-BMP antibodies suggesting an immunogenic reaction to the xenogenic BMP used (Gao et al., 1997). This again suggest that the sue of BMP infiltration of porous nitinol would be most valuable during the initial fracture healing stage post-implantation.

Reagent infiltration of NiTi has not yet bee examined. This group has infiltrated porous B₄ C+Al₂O₃ created with SHS with a bovine derived Bone Protein (Sulzer Orthopedics Biologics, Wheat Ridge, Colo.) in a rat skull on-lay model. Histologic analysis, bone ingrowth and surface contact measurements are currently being conducted. We are also currently in the process of implanting infiltrated porous NiTi using the same methods.

Biocoating of NiTi is an option for improving bony apposition. The surface characteristics of an implant play an important role in the rate and degree to which bone will bond with an implant (Kieswetter et al., 1996). Additionally, theoretical work has been done on how implant characteristics affect protein resorption. Human plasma Fibronectin (pFN) has been bonded to NiTi (Endo 1995). This coating promoted fibroblast spreading in an in vivo system along with decreased implant corrosion (Endo 1995). This modification offers a means to control or indeed reduce biological interactions with NiTi, with the possibility of making biocompatible materials bioactive, better mimicking the physiologic conditions. Biocoating in conjunction with reagent infiltration may be the best method of increasing, both bone ingrowth and apposition during the initial phases of bone development in the porous implant.

Reagent infiltration, biocoating or the combination of the two may offer the opportunity to expedite the biological fixation of NiTi to bone. These methods may also cause bone infiltration into deeper pores and stimulate bone maturation and general health. Improving the biological behavior of porous metallic implants like NiTi can ultimately create a highly effective material for bone replacement.

There is, most likely, no one material or implant architecture that may be considered the ultimate bone replacement material. One must be cognizant of the application of the material. including its location in the body and subsequent loading environments. Porous NiTi does appear to be sufficiently versatile as a material to warrant its consideration in bone engineering.

The potential for modification of NiTi's surface properties to create a bioactive implant is further encouragement.

### Example II

### The Interaction Between Bone and Porous Biomaterials in Rabbit and Human Craniomaxillofacial Bone

A. Porous Biomaterials in Craniomaxillofacial Applications

Surgery to repair defects in the skeleton surrounding the brain (crania) and face is becoming increasingly refined. Skeletal defects can be the result of heredity (e.g. craniosynostosis, craniocleidodysostosis), infection (pyogenic; and nonpyogenic osteomyelitis) or trauma (e.g.-segmental nonunion). In the repair of the bone, gaps can be created which must be filled to maintain the cosmesis of the bony structure. As a consequence, biomaterials other than autologous bone are being examined to fill these gaps and provide a scaffold upon which new bone can grow. The need to characterize subsequent biologic and mechanical interactions between these materials and bone in vivo is paramount given that, in clinical use, an implant may be in vivo for extended periods.

The advantage of porous materials, in general, is their ability to provide biologic fixation of the surrounding bony tissue via the ingrowth of mineralized tissue into the pore spaces. This is accomplished by increasing the available surface area for apposition (or bony contact) by having the interior of the implant accessible via pore spaces (Greene et al. 1997). It has been established that mineralized tissue ingrowth requires pore sizes in the range of 100-400 microns (Klawitter and Hulbert 1971; Hulbert et al. 1970). An open porosity (interconnected pores) allows for vascularization to support osseous tissue ingrowth and continued bone maturation (vanEeden and Ripamonti 1993). This architecture is analogous to the perpendicular aspects of bone morphology, exhibited at the vascular level by Haversian and Volkmann's canals. Interconnected pores increase stability and cosmesis of the bone (Kent and Zide 1984; Wolford et al. 1987) and increase resistance to fatigue loading (Epply and Sadove 1990). The increased stability reduces implant micromotion and the resultant resorption of adjacent bone (Kent and Zide 1984) or inhibition of cartilaginous ingrowth (Bragdon et al. 1996). Micromotion (or translational movement between the implant and bone) is movement under 150 μm (Bragdon et al. 1996; Ramamurti et al 1997). Implant morphologies such as described, allow for early rapid cartilaginous ingrowth and subsequent bone maturation over the lifetime of the implant.

Initial bone ingrowth into the implant porosity follows an ordered biologic progression similar to that of fracture healing. The first phase response to the implant is cellular in nature. Within minutes to hours, there is a rapid influx of undifferentiated tissue including mesenchymal and immune cells (histocytes) (Szachowicz, 1995). During the following 2-4 weeks fibroblasts in conjunction with capillary buds allow the implant pore space to be populated with preosteoblast cells. Osteoprogenitor cells from the periosteum and marrow, along with mesenchymal precursors, attach to the vascularized fibrous tissue subsequently differentiating along the osteoblast line. The osteoblasts: secrete osteoid that is then calcified, forming woven bone.(Kent and Zide, 1984).

This cellular response begins to wane and the second phase of healing begins in which the woven bone is replaced (remodeled) to subsequently form lamellar and Haversian type bone. This is accomplished by osteoclasts first removing the woven bone, forming a vascular channel that is lined with bone lining cells and osteoblasts. These cells secrete osteoid that is then calcified. The bone formed is known as lamellar or Haversian type bone (dependant upon whether a vascular channel exists). This second phase is open ended in its duration with remodeling occurring throughout the patient's life span.

An example of the progression bone into porous materials is seen in porous hydroxyapatite placed in the maxilla of humans. Significant amounts of woven bone is present in the pore space at 4 months up to 300 μm in depth (Ayers et al. 1999 Nunes et. al. 1997; Wolford et. al. 1987) The woven bone is then remodeled into lamellar bone over the subsequent 4 to 39 months with woven bone continuing to be formed as deep as 1500 μm into the implant and lamellar bone being prevalent at the interface and shallower regions of the implant. After 39 months Haversian type bone is prevalent with significant numbers of Haversian canals present; no woven bone and very little lamellar bone exists-after this time (Ayers et al.—1998). Bone ingrowth progresses until about 20 months reaching an asymptotic condition at all depths in the implant, with the relative amount of osseous tissue remaining constant (Ayers et al., 1999; Nunes et al. 1997). During this progression′, the bone matures into Haversian-based bone, exhibiting its normal structural properties and metabolism (Ayers et al. 1998). The HA, meanwhile, may undergo modest resorption (Nunes et al. 1997; Martin et al. 1993).

Porous Alloplastic Materials Used in Craniomaxillofacial Applications

The predominant implant materials currently in clinical use in oralmaxillofacial and craniofacial applications are autogenous bone, bank bone (such as antigen extracted autolyzed bone) and porous block hydroxyapatite (Interpore 200® is a commercial example of such a material in clinical use). Autogenous bone is the most common porous material used in craniofacial reconstruction (Phillips et al. 1992). Its use has the significant advantage of reduced rejection by the patient. Donor sites for autogenous bone include the rib, crania and iliac crest (Szachowicz 1995). Difficulties arise in the need for a secondary surgical site along with subsequent increases in operation time and the potential for donor site complications including, but not limited to infection, fracture and reduced patient ambulation (Kent and Zide 1984; Desilets et al., 1990; Motoki and Mulliken 1990). Bank bone may be used to eliminate the need for a second surgical site, but there still remains the disadvantage of potential improper bonding between the host bone and the graft and the possible infection (Kent and Zide 1984). Microhardness data indicates oven-ashed bone may provide an alternative (Broz et al. 1996). Nevertheless, the resorption rates of autogenous and allogenic bone grafts are unpredictable. As such, the possibility of early implant instability and failure remains (Kent and Zide 1984; Szachowicz 1995; Phillips et al. 1992). At its most optimum, a graft should be resorbed in such a manner that it allows sufficient time and structure for vascularization of the porosities and subsequent bone ingrowth (Phillips et al. 1992).

Slow resorption of the implant material is a reason that ceramic biomaterials based on calcium phosphates (the mineral phase of bone) have gained favor. These materials include hydroxyapatite (HA) and tricalcium phosphate (β-TCP). They can be manufactured to provide for controlled resorption with appropriate porosity (Eggli et al. 1987; Kent and Zide 1984; Light and Kanat 199 1). Ceramics have the disadvantage of being brittle and difficult to machine, however, they are strong enough to withstand the forces induced during mastication (Wolford et al. 1987; Holmes et al. 1988). Dense hydroxyapatite in the form of porous block coralline HA is an effective material for use in craniofacial applications (Ayers et al. 1998; Nunes et al. 1997; Wolford et al. 1987; Holmes et al. 1988; Jahn 1992). Sintered or plasma sprayed HA can be used as a porous coating for otherwise nonporous materials such metals (e.g. Ti6A14V titaniun,), providing a large area for micromechanical fixation via osseointegration of the implant, increasing its stability during the early phases of bone ingrowth (Engh and Bugbee 1998; Ducheyne 1998).

Porous NiTi as a Material for Bone Engineering

None of the metals in current use in craniomaxillofacial applications (e.g. Ti6Al4V titanium, CoCr, ASTM 316L Stainless Steel) are manufactured in porous forms. The surfaces of these materials can be made porous as mentioned before by the plasma spraying or sintering of ceramic or metallic beads to the surface. Manufacturing techniques such as double sintering and self-propagating-high-temperature-synthesis have allowed the production of completely porous metals such as porous, equiatomic NiTi shape memory alloy (approximately equal atomic masses of nickel and titanium). This material is undergoing consideration for use in craniofacial procedures (Simske and Sachdeva 1995; Ayers et al. 1999). The utility of nitinol as a superelastic, shape-memory alloy implant material has yet to be fully-investigated. In Russia, China and Germany, it has been in clinical use for approximately a decade in maxillofacial surgeries and other orthopedic procedures involving thousands of patients (Shabalovskaya 1996; Dai 1996; Airoldi and Riva—1996).

Porous nitinol can be produced by various manufacturing processes, including, but not limited to, sintering of molten NiTi and self-propagating-high-temperature-synthesis (SHS) (Itin et al. 1994; Yi and Moore 1990). Such methods allow for a controlled range of NiTi porosity creating a implant morphology similar to bone. 50% porous NiTi provides greater initial bone ingrowth (as a percentage of the implant cross-section) than 30% porous hydroxyapatite, primarily due to the greater exposed surface area (Simske and Sachdeva 1995). Moreover, NiTi in this porosity range provides a void space, after bone ingrowth, similar in percentage of cross-section to that of rabbit cranial bone further indicating NiTi's ability to at least architecturally mimic bone (Simske and Sachdeva 1995). The shape memory property of NiTi also allows for the possibility of in situ implant shape in the case of injury to the implant or surrounding hard tissue.

The superelasticity and high strength material properties of nitinol also suggest its candidacy for orthopedic implantation. The superelastic properties allow the surgeon greater margin in sizing bony defects as the implant can be press-fitted into the bone without unduly damaging the surrounding bone or implant. In fact, such a press fitted superelastic, shape-memory alloy may naturally space surrounding bone through cyclic resorption of the surrounding bony structures. The high Strength of NiTi (UTS of 895 MPa, annealed) allows for good initial fixation of the implant by withstanding the stresses induced by mastication or other imposed loads. With the incorporation of porosities into the NiTi, the potential for the matching of the mechanical properties of the implant to the surrounding bone becomes available, decreasing the prevalence and magnitude of subsequent stress-shielding.

Metals and ceramics in current clinical use have a modulus of elasticity in the range of 100-400 GPa. This is in contrast to bone, which has an elastic modulus an order of magnitude less (20 GPa for cortical bone with approximately ⅔ mineral mass percentage of dry mass). The martensitic modulus of elasticity for solid NiTi is in the 28-41 GPa range (close to the modulus of bone). By making NiTi 50% porous, the apparent modulus of the implant is below the range of bone (14-20 GPa). If an exact match between. a bone infiltrated implant and the surrounding bone is required to minimize stress-shielding, the low modulus of porous NiTi allows the possibility of significant ingrowth at this matching value. [tin et al. demonstrated further the ability of NiTi to mimic the mechanical properties showing 40-50% porous nitinol has a recoverable strain of 3.2% near physiologic temperatures, which is similar to the recoverable strain of bone at 2% (Itin et a]. 1994). This important aspect of NiTi superelasticity suggests that if the surrounding bone is strained within its elastic region (less than 2%), the implant will deform with the bone and recover its original shape afterwards, preserving the implant/bone bond. NM Biocompatibility Numerous studies have examined the biocompatibility of NiTi in vitro and in vivo, with differing results. Rondelli, using human body simulating, fluids reported that NiTi has a localized corrosion resistance similar to Ti6Al4V, but when the passivation layer is abruptly damaged, NiTi's corrosion resistance is less than Ti6Al4V while is still being comparable to other austenitic steels (such as ASTM 316L) (Rondelli 1996). Putters et al., using the inhibition of mitosis in human fibroblasts cultured on nitinol, titanium and nickel substrates, stated that the results indicate that nitinol is comparable to titanium in its biocompatibility (Putters et al. 1992). Sarkar et a]. showed that NiTi bad an earlier breakdown of its passive oxide layer than other implant materials such as titanium, stainless steel and cobalt-chrome alloys when subjected to potentiodynamic cyclic polarization tests in a sodium chloride solution (Sarkar et al. 1983). It should be noted, that these studies focused on the surfaces of solid NiTi; thus, it may be expected that porous NiTi may have diminished corrosion resistance by the fact of its greater surface area in contact with bodily fluids.

In vivo work is generally supportive of NiTi's biocompatibility. Simske and Sachdeva, and more recently Ayers et al. (1999) have demonstrated that bone ingrowth into porous nitinol in the crania of rabbits is evident as early as six weeks and that bone contact is made with the surrounding crania] hard tissue (Simske and Sachdeva 1995; Ayers et al. 1999). A study using high purity nitinol alloy implanted in the femurs of beagles for 3, 6, 12, and 17 months showed no evidence of localized, or general corrosion on the surfaces of the implants and no metallic contamination of organs due to the implants (Castleman et al, 1976). Using quantitative histomorphometry, nitinol was shown to be progressively encapsulated by bony tissue in the tibiae of rats, albeit at a reduced rate when compared to pure titanium, anodic oxidized Ti and Ti6A14V, over the course of a 168-day experimental period (Takeshita et al. 1997). In a finding similar to Takeshita et al., Berger-Gorbet et al., using immunohistochemistry, showed NiTi screws implanted in rabbit tibia had-slower osteogenesis with no close contact between implant and bone as compared to screws made of commercially pure titanium, Vitallium, Duplex austenitic-ferritic stainless. steel (SAF), and 3 16L Stainless Steel (Berger-Gorbet et al. 1996). Clinical results of procedures using NiTi alloys in China and Russia state no significant detrimental effects of devices implanted in craniofacial bone (Shabalovskaya 1996; Dai 1996). However, the specific studies upon which this conclusion is made are not readily obtainable, making replication difficult.

Considerations for Application of Porous Biomaterials

Mechanical Considerations

One of the primary concerns of bone engineering arises from the premise of “Wolff's Law”: that bone not subjected to loading undergoes net resorption. When an implant with an elastic modulus stiffer than bone is used, mechanical disuse causes the surrounding bone to resorb (stress-shielding), threatening the stability of the implant. Thus, matching the material properties of the implant to the bone for a given appreciation may be paramount to the success of a porous metal implant. Material, property matching is perhaps less important in craniofacial applications than in joint replacement (e.g. hip and knee arthroplasty), due to the different mechanisms governing bone growth. (Rawlinson et al. 1995). Nonetheless, the mechanical aspect of craniofacial implantation must be considered (Ayers et al., 1999).

It would be inappropriate to assign a single value to the elastic modulus of solid NiTi because the elastic modulus is nonlinear with respect to temperature. The martensitic elastic modulus follows the Clausius-Clapeyron equation in the form of ∂σₐ/∂Mₛ=−ΔH/Tε₀ where σₐ is the applied stress, Mₛ, is martensitic temperature, 60 is the transformation strain resolved along the line of the applied stress, ε₀ is the transformation latent heat and T is the temperature (Otsuka and Wayman 1998). Thus, there is a family of stress-strain curves dependent upon temperature for a given specimen. When porous, determining the structural modulus of the implant is further complicated. For example, at a temperature of 293° K, the modulus of 40-50% porous-nitinol is approximately 25 GPa (Itin et al. 1994). This compares to standard biomedical titanium alloys such as solid Ti6A14V with a modulus of 110 GPa. Other metals such as ASTM 3) 16L and CoCr alloys have elastic moduli of 200 and 220 GPa, respectively if they are solid. Roughly, the metals used in clinical applications are an order of magnitude stiffer than bone, while 40-50% porous NiTi is similar to bone in stiffness. The solid elastic modulus of bioceramic materials ranges from 40-117 GPa for pure crystalline hydroxyapatite to as high as 400 GPa for corundum, although these values can be reduced by upwards of an order of magnitude by the incorporation of porosities (Simske et al. 1997).

Formation Considerations

As stated previously, metals such as Ti6Al4V and CoCr are not normally manufactured in a porous form. They can be made “porous”, however, by coating the outer surfaces with metal powders via plasma spraying either metal or ceramic powders onto the metal surface; or by double sintering metallic beads onto the heated metal substrate, Pore sizes can range from 150-300 microns using these techniques with percent porosity form 20-40% (Simske et al. 1997). While porous coatings may enhance the osseointegration of the implant, it has been shown that the bond between bone and coating is preserved better than the bond between the coating and the substrate, resulting in the possible failure at the implant coating/substrate interface (Spector 1987; Vercaigne et al. 1998).

Ceramics occur naturally as porous materials (e.g. bone, coral, etc.) or can be manufactured to be porous via numerous methods including combustion synthesis, sintering, and plasma spraying. There are numerous ceramics in use today and it would be prohibitive to discuss them in this space. Rather, there are several extensive reviews available to the reader that discuss porous materials, including ceramics, for bone engineering (Simske et al., 1997, Bajpai and Billotte, 1995; Lakes, 1995). Suffice that ceramic materials lend themselves to formation in porous forms.

Implant elastic modulus values can also be adjusted based upon the natural or manufactured porosity of the materials. For example, the elastic modulus of corals, which are predominately hydroxyapatite, changes by an order of magnitude over a porosity range of 0-50%; thus, a 100 GPa modulus can be reduced to 10 GPa in a highly porous form (30-50%) (Simske et al. 1997). The apparent modulus of the porous forms of materials may be described via the equation E=Eₛ(Vₛ)^{x} where E is the apparent modulus, Eₛ is the elastic modulus of the solid; Vₛ is the volume fraction of the solid phase; X is a variable ranging from 1 to 2, being approximately 1 when Vₛ is approximately 1 and approximately 2 when Vₛ is approximately 0 (Lakes 1995). Given this, it is apparent that within an acceptable range of porosities, metallic, ceramic and glass materials can be manufactured to have apparent densities that of bone.

Machining

Machining considerations must also be taken into account when comparing these materials. This consideration arises from the need for the surgeon to be able to size the implant to the bony defect during the surgery to provide the best possible match between the implant and surrounding bone. Ceramics are very brittle, and are difficult to machine: warnings about the brittleness are prevalent in the literature. This is largely mitigated by the ability to form the ceramic into the appropriate shape beforehand, reducing the need for post-production machining. Porous metals formed by sintering or the plasma spraying of powders and diffusion bonding of metal fibers to a metal substrate can result in the damage to the underlying substrate and a coating that is also brittle and difficult to machine (Simske et al. 1997). Self-propagating-high-temperature-synthesis (SHS) has, nevertheless, allowed the manufacture relatively complex shapes in nitinol (cones, polygons, etc.) reducing the need for postproduction machining. The use of SHS in the formation of nitinol allows implants to be created very rapidly (on the order of seconds to minutes) in contrast to sintering or diffusion bonding processes, which can take hours to days to complete (Yi and Moore 1990).

Biocompatibilily

Ceramics and glasses such as HA, TCP and bioglasses are quite biocompatible. They promote the differentiation of the osteoblast phenotype from marrow stem cells, and are thus, osteoionductive in addition to being biocompatible. These materials are also osteoconductive in promoting the attachment of osseous tissue to the implant surface. Another advantage of these ceramics over metals such as nitinol is their ability to degrade over time, allowing bone to fill in the implant space. While the biocompatibility of NiTi is still under study, it has been the inventor's experience that NiTi is bioinert in vivo. It acts as an osteopermissive (or bioinert, similar to pure Ti and its alloys) material simply providing a scaffold upon which the bone may grow, neither promoting bone formation nor preventing it. As has been discussed earlier, the passive oxide layer can render NiTi bioactive similar to HA, TCP and bioglass. There does exist sufficient clinical evidence that over long-term implantation NiTi remains inert while metals such as ASTM316L Stainless Steel, which have been optimized for corrosion resistance (hence biocompatibility), will corrode.

Hypotheses

What may be determined from this introduction is that numerous factors affect bone ingrowth into porous implants. Some of these factors include, but are not limited to, the porosity of the implant material (pore size, pore gradient, percent porosity), the Lime of implantation, material biocompatibility, depth of porosity into the implant, implant stiffness, amount of micromotion between the implant and adjacent bone. The literature contains numerous studies that have examined the effects of material biocompatibility, implant micromotion, mechanical effects of implant stiffness on bone.

It is for this reason, the work presented in this dissertation seeks to elucidate the effects of porosity, time and depth into a porous craniofacial implant. Previous work has examined these factors only in a cursory fashion or as an aside to a specific hypothesis. The studies presented herein sought to examine the effects on bone ingrowth by these factors both individually and in conjunction with each other and quantify these effects.

The overall hypotheses set forth in this invention are as such:

1) Bone ingrowth into a porous implant is affected by pore size, time of implantation, and depth of porosity into the implant.

A) Pore size does not influence cranial bone ingrowth in the early post-implantation time frame (6-weeks).

B) Initial bone ingrowth is primarily a function of the biologic action of the tissues.

C) Given an appropriately sized porosity, bone continues to grow and mature within the pore spaces over extended periods of time.

D) Over time bone approaches an asymptotic value of ingrowth with the relative amount of osseous tissue remaining constant.

E) Bone ingrowth decreases with increasing depth into the implant.

F) Biologic factors are predominant at depths greater than 1 mm into the implant.

2) Bone ingrowth into porous craniofacial implants can be systematically quantified temporally and spatially.

B. Effect of Nitinol Implant Porosity on Cranial Bone Ingrowth and Apposition after 6 Weeks

Synopsis:

The present study addresses two aspects in the use of nitinol in cranial bone defect repair. The first is to examine the extent that porosity controls the early post-implantation cranial bone ingrowth. The second is to determine if 6-weeks is sufficient to measure significant bone ingrowth. In so doing, this study tests the hypothesis that pore size of an implant does not influence early cranial bone ingrowth during the initial healing phase after implantation (hypothesis 1 A). This begins to establish at what point in time in vivo porosity control of craniofacial bone ingrowth may occur.

Porous equiatomic (equal atomic masses of titanium and nickel) nickel-titanium (nitinol) implants with three different morphologies (differing in pore size and percent porosity) were implanted (6 weeks) into the parietal bones of New Zealand White rabbits. Ingrowth of bone into the implant and apposition of bone along the exterior and interior implant surfaces was calculated. Mean pore size (MPS) of Implant Type #1 (353+/−74 μm) differed considerably than Implant Type #2 (218 28 μm), and Implant Type #3 (178+/−31 μm). There was no significant difference between implant types in the percentages of bone and void/soft tissue composition of the aggregate implants, The amount of bone ingrowth was also not significantly different between implant types. Implant #1 was significantly higher in pore volume and thus had a significantly higher volume of ingrown bone (2.59 0.60 mm³) than Implant #3 (1.52+/−0.66 mm³) and a greater amount, but not significantly, than Implant #2 (1.76+/−0.47 mm³). Pore size does not appear to affect the bone ingrowth during the cartilaginous period of bone growth in the implant, This implies that over the commonly accepted range of implant porosities (150-400 μm) the bone ingrowth near the interface of nitinol implants at six weeks is similar.

Introduction

Alloplastic implants have long been considered for repair and replacement of bone in craniofacial applications to provide for the structure, mechanical properties and cosmesis of the bone (Wolford et al. 1987; Jahn 1992; Nelson et al. 1993). Porous, alloplastic implants have an advantage over autogenous grafts in avoiding implant resorption and allowing sufficient time for structural stabilization of the implant. In addition, they eliminate the need for a donor site with its concomitant increases in operation time and the potential for donor site complications including, but not limited to, infection and fracture (Desilets et al. 1990; Motoki and Mulliken 1990; Goldberg et al. 1993). Current alloplastic materials in clinical use for craniomaxillofacial repair and reconstruction include hydroxyapatite (Wolford et al. 1987), calcium phosphate (Nelson et al. 1993) and titanium (Ivanoff et al. 1997).

Porous nickel-titanium (nitinol) has been investigated as an orthopedic implant material for craniofacial applications (Simske and Sachdeva 1995). Porous nitinol offers the advantage of interfacial porosity as well as a permanent structural framework for the long-term replacement of bone defects. Moreover, the shape-memory characteristics of nitinol offer the possibility for in situ recovery of implant shape subsequent to any injury to the implant or surrounding hard tissue. An advantage of porous nitinol over other metals is that the porosity can be controlled, and an appropriately interconnected (open) framework of pore spaces can created for bone growth. Previous work has determined that porous implants must have interconnecting fenestrations to provide space for vascular tissue required for continued mineralized bone growth (Hulbert et al. 1970; van Eeden and Ripamonti 1994). This is a limitation in the manufacture of most metals used in bone engineering, but the manufacturing process used in the creation of the porous nitinol allows for a nearly 100% open pore structure (Itin et al. 1994).

Nitinol, like other titanium implant materials, is biocompatible (Shabalovskaya 1996) and corrosion resistant (Shabalovsaya et al. 1994). Its porosity can be controlled over a range of 8-60%, resulting in a 0.2-1.0 GPa range for strength limit, and 5-200 MPa range for yield strength (Itin et al. 1994). Nitinol is low in corrosion due to the formation of a natural passivation oxide (Melton and Harrison 1994; Oshida et al. 1992), and has been used in maxillofacial surgeries in more than 1400 patients (Sysolyatin et al. 1994). In a previous investigation, nitinol was found to provide substantial bone ingrowth and apposition in rabbits by 6 and 12 weeks post-implantation (Simske and Sachdeva 1995).

The present invention addresses two aspects of the use of nitinol in craniomaxillofacial surgery. The first is to verify if six weeks' time is sufficient for substantial bone ingrowth into and apposition against the implant. The second is to determine the effect of pore size on the ability of bone to grow into the implant during the early (6-week) post-operative period.

Methods and Materials

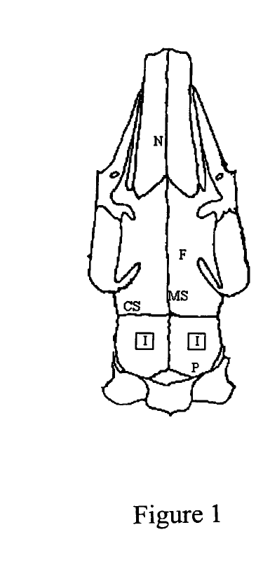

National Institutes of Health (NIH) guidelines for the care and use of laboratory animals (NIH publication 85-23 Rev. 1985) were observed throughout the experiment and institutional animal care and use committee approval was obtained before any surgeries were performed. Porous, equiatomic (equal atomic masses of titanium and nickel) nickel-titanium (nitinol) implants with three different morphologies (differing in pore size and percent porosity) were implanted into the parietal bone of New Zealand White rabbits (2.5 months old-Hazelton) (FIG. 1). The implants were machined to 5 mm×5 mm squares (thicknesses from 305-676 μm). After machining, the implants were autoclaved (120° C.) for 30 minutes to ensure their sterility. The scalp overlying the frontal, parietal and occipital cranial bones of ten rabbits was shaved in preparation for the implant surgeries. The animals were anesthetized (xylazine, 10 mg/kg, SC; ketamine, 50 mg/kg, IM), and sterile surgical techniques were used to fold back the dermal and subdermal layers to expose the underlying periosteal connective tissue layer. This layer was carefully sectioned, exposing the parietal cranium. Underlying cranial bone on one side of the midsagittal suture caudal to the coronal suture was removed to the depth of the implant thickness using a stereotaxic drill and burr bit, irrigated with sterile saline (0.9% wgt/vol). The shape and depth of the defect was checked against the implant (5 mm×5mm square) prior to the implant placement. This was repeated for implant placement in the parietal bone on the opposite side of the midsagittal suture. The implants were assigned such that samples from two of the three implant groups were placed in each rabbit. Thus all combinations of implant pairing (e.g. Type 1 & Type 2, Type I & Type 3, Type 2 & Type 3) in the craniums were represented in n=3, n=4, and n=3 rabbits respectively. The implants were embedded (press fitted by hand only) and the periosteal layer sutured back into position. The rabbits were allowed to recover from the surgeries for 6 weeks. Twenty implants were thus placed into rabbit cranial bone: 7 of Implant #1, 6 of Implant #2, and 7 of Implant #3 (characteristics described below). Test surgeries using 2 rabbits, for a total of 4 implants, used the surgical procedure outlined above but bonded the implants to the underlying bone with surgical cyanoacrylate which delayed bone ingrowth by approximately 2 weeks.

The implantation time was chosen to allow comparison of ingrowth and apposition among implant types during cartilaginous bone ingrowth (Spector 1982; Ripamonti 1991; Ripamonti et al. 1993). Thus, a six-week implant time was chosen. After this length of time, the rabbits were killed using xylazine (10 mg/kg, SC) followed by an overdose of carbon dioxide. The frontal and parietal bones of the rabbit cranium were removed as a unit, and separated from surrounding soft tissue with a scalpel. The bones were immediately placed in neutral (pH=7.0) buffered (Dulbecco's phosphate buffered saline, PBS) 10% formalin for 48 hr, then rinsed and stored in 70% ethanol until histology was performed.

Before histology, the sections were rinsed of the 70% ethanol with PBS, then cleared. of excess bone to a 2 mm border around the implants with a manual high-speed saw (Dremel). Each implant was embedded in Epo-Kwick epoxy resin (Buehler, Lake Bluff, Ill.), then sectioned transversely (Isomet saw, 300-micron thick blade, 10.2-cm diameter) at increments of 0.5, 1.8, 3.1, and 4.4 mm along the length; exposing four surfaces with the dimensions of 5 mm by the implant thickness. Each surface was wheel-polished (600-grit silicon carbide paper), smoothed (6-micron diamond paste) and stained (0.1% formic acid, 3 minutes; 20% methanol, 120 minutes; toluidiine blue solution [1% wgt/vol toluidine blue; 1% wgt/vol sodium tetraboratel] 2 minutes) to provide blue coloring to the collagen phase of the bone. The sections were illuminated under far blue (405 nm) light. Photomicrographs were taken (Carl Zeiss, Inc., Axioskop/MC80 camera mount), developed and pieced together to provide highly magnified (112×) cross-sections (typically 56 cm in width, and 3.4-7.6 cm in height) for the quantitative histomorphometric analysis.

Quantitative histomorphometry was calculated for the implant surface (implant/bone interface) and within the implant. Each section (4 total/implant) was overlaid with a transparent grid with pseudo-random “hits” placed at a density of approximately 1 hit/cm², thus providing 120-130 hits along the implant/bone interface per exposed section (600-650 per implant), and 190-420 hits within the implant per exposed section (950-2100 per implant). Additionally, hits along the interface of the interior of the implant (500-1500 per implant) were counted (indicating the extent of surface contact of ingrown bone within the implant). Each hit along the interface (the exterior and interior interfaces were separately counted) was classified as either bony apposition (I_{b}) or soft tissue or void (void is present, in part, due to disproportionate shrinkage of the soft tissue) apposition (Iₛₜ). Within the implant, each hit was classified as one of the following: bone (W_{b}), soft tissue or void (Wₛₜ), or implant (Wᵢ) material. Ratios of these counts (I_{b}, Iₛₜ, W_{b}, Wₛₜ, Wᵢ) multiplied by 100%, were used to determine histomorphometric parameters of interest (Table IV).

TABLE IV

| Parameter | Ratio | Interpretation |
|---|---|---|
| Percent Implant | Wᵢ/(Wb + Wₛₜ + Wᵢ) | Percent of the implant cross-section that is implant |
| Percent Void | Wₛₜ/(W_{b} + Wₛₜ + Wᵢ) | Percent of the implant cross-section that is void |
| Percent Bone | W_{b}/(W_{b} + Wₛₜ + Wᵢ) | Percent of the implant cross-section that is bone |
| Percent Ingrowth | W_{b}/(W_{b} + Wₛₜ + Wᵢ) | Percent bone in available ingrowth space in implant |
| Bony Apposition | I_{b}/(I_{b} + Iₛₜ) | Percent bone apposition against the implant surface |
| Histomorphometric parameters of interest determined from ratios of “hit” counts. W_{b}, Wₛₜ and Wᵢ indicates the number of “hits” for bone, soft tissue/void, and implant material, respectively, within the implant. I_{b} and Iₛₜ indicate the number of “hit” counts for bone and soft tissue/void | | |
| #apposition, respectively. Bony Apposition, I_{b}/(I_{b} + Iₛₜ), was determined for both the exterior interface of the implant, and separately the interior interfaces (along the pore linings). | | |

Additional parameters were calculated for each implant. Each implant's thickness was measured at 25 equally spaced locations, and the mean value used to compute the implant thickness (in microns) and implant volume (in mm³, determined by multiplying the thickness by the implant area, 25 mm²). The available volume for bone ingrowth was calculated as (100%—Percent Implant) multiplied by the implant volume. This value, multiplied by the ratio W_{b}/W_{b}+Wₛₜ) allowed the calculation of total volumetric bone ingrowth (in mm³) into the implant. Finally, all visible pores in the implants were measured in the 2-dimensional micrographs, and their values averaged for each implant (typically 40-50/implant). Pore size measurements were taken at the interface and were corrected by 4/π to obtain their three-dimensional diameters (Parfitt et al. 1987).

Finally, simple geometric measurements for the implants were obtained. Total 2 surface area was obtained from the equation: (50+20*Implant Thickness) mm , where the 50 mm² is from the two, 5 mm×5 mm surfaces, and the (20*Implant Thickness) from the four (5 mm×Implant Thickness) mm² surfaces. Total surface porosity was obtained by multiplying the total surface area by (100%—Percent Implant).

Statistical comparisons of measurements among the three implant types were performed using analysis of variance (ANOVA), followed by the Tukey-Kramer HSD (honestly significant difference) to determine group-group differences. A 95% level of significance (α=0.05) was used for the Tukey-Kramer HSD (JMP, SAS Institute Inc).

Results

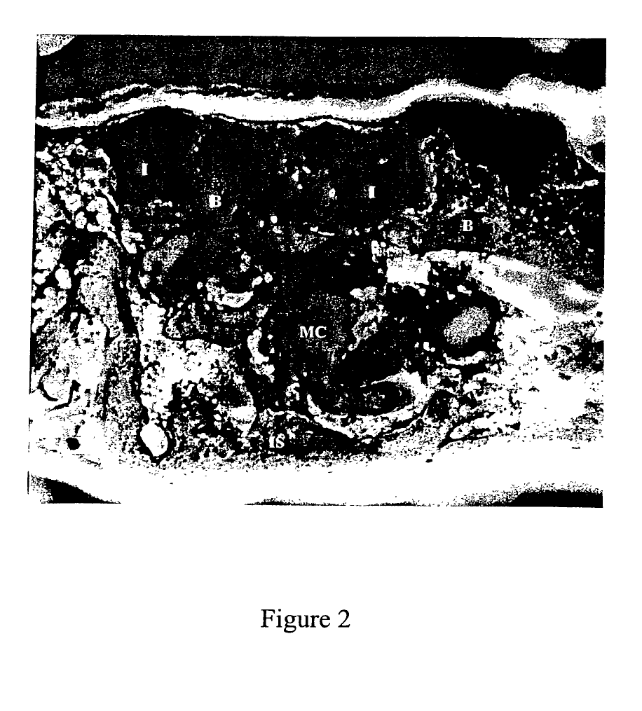

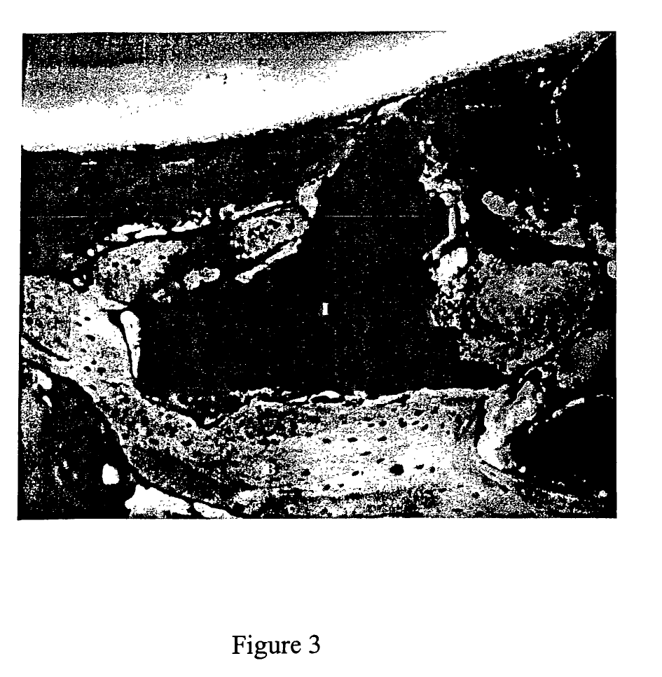

Microscopic examination showed evidence of fibrovascular tissue influx and concomitant bone formation in the pore spaces of all implants (FIGS. 2 and 3).

The bone present in the pore spaces was of the woven type. Vascular buds were discemable within both the pore spaces and the interconnecting fenestrations. No apparent inflammatory response was noted.

The implant types were significantly different in thickness. Implant# 1 was thickest, resulting in a significantly larger overall implant volume than Implant #2 and Implant #3 (Table V).

TABLE V

| Measurement | Implant 41 (n = 7) | Implant #2 (n = 6) | Implant #3 (n = 7) |
|---|---|---|---|
| Thickness (μm) | 644 +/− 21* | 345 +/− 37 | 385 +/− 56 |
| % Volume Pore Space (Porosity) | 42.9 +/− 4.0* | 54.4 +/− 5.3 | 50.5 +/− 13.7 |
| Mean Pore Size (μm) | 353 +/− 74* | 218 +/− 28 | 179 +/− 31 |
| Available Pore Volume for | 6.91 +/− 0.61* | 4.67 +/− 0.26 | 5.10 +/− 1.96 |
| Ingrowth (mm³) | | | |
| Implant thickness, percent porosity, mean pore size and available volume for bone ingrowth given as N_ =/− standard error of the mean for each of the three implant types. Mean pore size is the mean of all measurable pores for a given implant. Pores are defined as the openings into the implant at the surface of the implant. | | | |
| #.*Denotes measurements statistically significantly (P < 0.05, Tukey-Kramer HSD) different in Implant #1 when compared to either Implant #2 or Implant #3. | | | |

Differences in pore structure were confined to Implant #1, with a significantly larger average pore size (353+/−74 μm) than Implants #2 (218 28+/−28 μm) or #3 (179+/−31 μm) (which were not significantly different from each other). Because the implants were machined to the same 5 mm×5 mm dimensions, Implant #1, due to its greater thickness, had a significantly greater available pore volume for bone ingrowth (6.91 0.61 mm 3) than either Implant #2 (4.67+/−0.26 mm³) or Implant #3 (5.10+/−1.96 mm³). Although Implant #1 had a higher pore volume, its porosity was the lowest of all three implant types (43%, 54%, 50%, Implant #1, #2, #3 respectively),

Quantitative histomorphometry of ingrowth showed few significant differences between implant types. There were no significant differences between implant types for the percent bone (14.6+/−5.9%, 20.8+/−6.7%, 15.4+/−4.7%, Implant #1, #2, #3 respectively) or percent void (26.9+/−3.8%, 33.6+/−5.1%, 35.1+/−10.9%, Implant #1, #2, #3 respectively) (Table VI).

TABLE VI

| | Implant #1 | Implant #2 | Implant #3 |
|---|---|---|---|
| Measurement | (n = 7) | (n = 6) | (n = 7) |
| Percent Implant (%) | 57.1 +/− 4.0 | 45.6 +/− 5.3 | 49.5 +/− 13.7 |
| Percent Void (%) | 26.9 +/− 3.8 | 33.6 +/− 5.1 | 35.1 +/− 10.9 |
| Percent Bone (%) | 14.6 +/− 5.9 | 20.8 +/− 6.7 | 15.4 +/− 4.7 |
| Percent Ingrowth | 37.4 +/− 7.8 | 37.9 +/− 10.1 | 31.1 +/− 6.9 |
| (%) | | | |
| Bony Apposition, | 47.4 +/− 9.6# | 41.6 +/− 9.2 | 32.0 +/− 9.1 |
| Exterior (%) | | | |
| Bony Apposition, | 38.6 +/− 12.7 | 41.9 +/− 10.5 | 36.0 +/− 11.1 |
| Interior (%) | | | |
| Total Bone | 2.59 +/− 0.60# | 1.76 +/− 0.47 | 1.52 +/− 0.66 |
| Ingrowth (mm³) | | | |
| Ingrowth and apposition characteristics (defined in Table VI) and total volumetric bone ingrowth for the three implant types. Total bone ingrowth is obtained by multiplying Percent Ingrowth (Table VI) by Available Pore Volume for Ingrowth (Table V). Values are given as mean +/− standard error of the mean for each of the three implant | | | |
| #types. An asterisk (*) indicates a significant difference (P < 0.05) from Implant #2. A pound sign#indicates a significant difference (P < 0.05) from Implant #3. | | | |

The bone ingrowth into the available pore space, Percent Ingrowth W_{b}/(W_{b}+Wₛₜ)), showed no significant differences between implant morphologies. The greater available pore volume for bone ingrowth, in conjunction with a % Bone similar to the other two implant types, resulted in Implant #1 having a significantly higher volume of ingrown bone (2.59+/−0.60 mm³ than Implant #3 (1.52+/−0.66 mm³) and higher volume, although not significant, than Implant#2 (1.76+/−0.47 mm³). This is despite modest (10%) differences in total surface area (62.9 mm² mean for Implant#1, 56.9 mm² for Implant #1, and 56.9 mm² for Implant #2, and 57.7 mm² mean for Implant #3) among the implants, and less total surface porosity (27.0 mm² mean for Implant #1, 30.7 mm² for Implant #2, and 28.9 mm² mean for Implant #3) for Implant #1.

Significant differences in apposition measurements were confined to the exterior apposition of Implant #1 (47.4+/−9.6%) and Implant #3 (32.0+/−9.1%). The interior apposition measurements and the difference between exterior and interior apposition did not vary significantly among implant types.

Discussion

Microscopic examination showed that all implants had early bone ingrowth. Bone within the pore spaces was primarily of the woven type. Vascular buds were observed in the pore spaces and interconnecting fenestrations. No giant cell (macrophage) reaction was noted, as in a previous study (Simske and Sachdeva 1995). Thus, from a gross microscopic perspective, the nitinol implants demonstrated biocompatibility.

The six-week time-period is sufficient to measure significant ingrowth of immature bone into the pores of these thin nitinol implants. The bone ingrowth into the implants bonded with surgical cyanoacrylate also contained measurable amounts of ingrowth (12-23% range, n=2 Implant #1, and n=1 for both Implant #2 and #3), albeit at reduced levels, after 6 weeks. Ingrowth and apposition measurements for all implant morphologies were significant and similar to values measured previously (Simske and Sachdeva 1995). Other recent work has suggested a slower osteogenic process in adjacent bone when nitinol screws were placed in the tibia of rabbits (Berger-Gorbet et al. 1996). A long-term study (>12 weeks) may be appropriate to determine if the values measured here increase slowly over time (indicating the slower bone development noted); or diminish as remodeling occurs and are thus artifacts of the initial immune response (Motoki and Mulliken 1990; van Eeden and Ripamonti 1994) or the chondrocyte-mediated initial bone ingrowth to the implant (Parfitt et al. 1987).

There is no apparent correlation between pore size in these thin implants and the amount of bone ingrowth. The lack of significance difference in bone ingrowth between implant types may be due to the use of thin implants (their thickness is of the same order as the pore size). This may imply that a minimum thickness to porosity ratio is required in order to measure pore size effects on bone ingrowth. Manufacturing constraints limited the availability of thicker “plate” type material for this study. During surgery, press fitting the implants into the bone may cause sufficient osteogenic material to be integrated throughout the pore spaces so that bone ingrowth occurs regardless of pore size. Other previous studies have shown that significant ingrowth into porous implants of full cortical thickness occurs in pore sizes as small as 75-100 μm and as early as 4 weeks (Klawitter and Hulbert 1971). In that same study, ingrowth was also observed as deep as 600 μm in porous implants with pore sizes ranging from 175-200 μm after 11 weeks. Because the smallest pore size used in this study is of the same order as the largest pore size used by Klawitter and Hulbert, the significant bone ingrowth observed for all implant morphologies in this study is not unexpected. Hence, the implants used here may be considered similar to a porous coating of the interface portion of a larger implant when apparently, above 150-200 μm, increased porosity is unnecessary to enhance early, cartilaginous ingrowth.

Not using surgical glue for attaching the implants may have possibly contributed to the unsuccessful implantation of 1 of the 20 implants (implied by a higher interior than exterior apposition). However, its apposition measurements suggest that the implant was biocompatible. Previous studies have used the measurement of the percentage of bone in contact with the implant as a determinant of biocompatibility for both hydroxyapatite (HA) and apatite-wollastonite glass ceramic (A-W G.C) (Neo et al. 1998; Ono et al. 1990). Ono et aL showed that A-W G.C granules were 90% covered by newly formed bone and HA granules were covered by 60% of new bone suggesting that A-W G.C is more osteoconductive than HA. Neo et al. examined the cellular function of the osteoblasts lining these materials further elucidating their osteoconduction. A recent study using titanium bars placed in the tibia of goats showed that untreated titanium had a mean bone-to-implant surface contact of 14% (Vercaigne et al. 1998). The samples in this study averaged 40% for both exterior and interior apposition, suggesting that while nitinol may not be osteoconductive, it is osteopermissive. The findings here are similar to those by Takeshita et al. where it was observed that nitinol was progressively encapsulated by bone over the 168 day study (Tekshita et al. 1997). These findings are in contrast to a recent study by Berger-Gorbet et al. in which no close bone contact to nitinol screws in rabbit tibia was noted over the course of 3, 6, and 12 weeks of implantation time (140X original magnification) (Berger-Gorbet 1996).

This study provides further evidence that -porous nitinol is capable of supporting early ingrowth of bone into the implant. Considerable bone ingrowth and apposition were observed for all three of the implant types. This implies that the peak bone ingrowth near the interface of nitinol implants at 6-weeks is similar over the normal range of implant porosities (e.g. 150-400 μm). In craniofacial applications, an implant consisting of a porous ceramic with a thin nitinol interface may provide a scaffold that supports early bone ingrowth, implant fixation, and limit stress-shielding. A functionally graded interface between the nitinol and ceramic improves the elastic properties of the ceramic while decreasing ceramic brittleness (Itin et al. 1997). Such an implant would then allow modulus matching between the bone and implant. Subsequent investigations, using plate material, on the effects of pore size range, pore distribution, and pore shape (i.e., interior to the implant) are necessary to help fully develop the capabilities of nitinol implant design.

C. Long-Term Bone Ingrowth and Residual Microhardness of Porous Block Hydroxyapatite Implants in Humans Synopsis

Since pore size has little effect on the early cranial bone ingrowth, it was apparent that the most effective method to develop and understanding of the various overall mechanisms governing craniofacial bone ingrowth (e.g. bone biology, pore size, time of implantation). This was accomplished by quantitative histomorphometrical measurements on craniofacial bone ingrowth into a clinically accepted porous implant over short and long time periods. In so doing, a better understanding of when the aforementioned mechanisms come into play during the time of implantation is developed. This study tests the hypotheses that given an appropriately sized porosity, craniofacial bone continues to grow into and mature within the pore spaces over extended periods (hypothesis 1C) and that craniofacial bone ingrowth asymptotically approaches a value of ingrowth with the relative amount of osseous tissue remaining constant (hypothesis 1D).

Twenty-five maxillary HA implants (4-138 months of implantation, mean 32 months) were removed from 17 patients. These implants had been placed into the lateral maxillary wall, juxtapositioned to the maxillary sinus during orthognathic surgery, and were harvested for analysis after voluntary consent. Microscopic examination showed normal bone morphology in all implants; no inflammatory response was observed. Histomorphometric measurements indicated that there was significant bone ingrowth in all implants, with an overall mean of 23±7% bone (range, 7-31%), 51±7%HA matrix (range, 39-65%), and the remainder being soft tissue or void at 26±9% (range, 10-40%). No significant difference in microhardness values between the bone in the implant and the bone surrounding the implant was noted, indicating the structural integrity of the porous block HA/bone aggregate had been maintained. Bone ingrowth appeared to plateau around 20 months, reaching an equilibrium in which the relative amount of osseous tissue remained constant. Based on the findings in this study, porous block hydroxyapatite is a viable material for long term implantation to the maxilla in orthognathic surgery.

Introduction

Bone repositioning during craniofacial reconstruction and orthognathic surgery frequently results in gaps that must be filled to maintain structural stability and bony continuity (Wolford et al. 1987). Implants used for this purpose must be able to withstand the stresses induced through mastication (Hiatt et al. 1987). In addition, the graft must be biocompatable and encourage connective tissue and bone ingrowth to become fully incorporated into the existing bone (Holmes et al. (1988).

Porous materials and coatings have been investigated extensively in oral and maxillofacial surgical literature. Porous materials offer the advantage of cementless, biologic fixation via bone ingrowth into the interconnecting pores (Spector 1987; Klawitter and Hulbert 1971; Simske et al. 1997). This continuum of ingrowing bone and implant results in increased ability to withstand fatigue loads over nonporous implants (Eppley and Sadove 1990).

This study describes the long-term ingrowth of bone into human HA implants. Microhardness was used as a measure of bone mineralization and structural properties, as well as a measure of potential implant degradation.

The Use of Microhardness as a Measure of Material Properties

Microhardness provides an approximate measure of the material properties of a material. Hardness is defined as a material's ability to resist penetration by an indenter. Microhardness measures this at a level roughly defined as an indent of less than 100 μm under a load of less than 200g (Evans et al., 1990; Amprino, 1958). Nanohardness refers to indentation that is too small to be resolved with optical microscopy and provides material measurements at a molecular level (Reister et al., 1998).

Incomplete or abnormal bone formation can lead to subsequent implant instability and failure. Microhardness is the most readily obtainable measurement of bone material properties within pore spaces. The size of the indenter allows for access to pore spaces as small as 100 μm in diameter. Thus, it allows for direct measurement of material properties within the pore space, without significant damage to the specimen. While the absolute values of the measurements made using this technique are subject to interpretation, they do allow for comparison of the same measurements made at other sites. Hardness measurements are primarily applicable to compact bone (Currey and Brear, 1990). Other properties such as porosity, collagen orientation may also affect these measurements (Currey and Brear, 1990).

Microhardness is nearly linearly correlated to Young's modulus and calcium content in mammalian mineralized tissue, thus suggesting that microhardness can be an indicator of local, bone structural integrity (Houde et al. 1995, Currey and Brear, 1990). These correlations were developed empirically by Currey and Brear (1990). They utilized various mammal species of varying ages to obtain a range of values for Young's modulus. The Young's modulus of each sample was determined using machined specimens loaded to failure in tension while calcium content was determined calorimetrically from a small amount of material taken from the region of the indentation fracture surface (Currey and Brear, 1990). These findings validate the results obtained by Hodgskinson et al. (1989), using the proximal end of the bovine femur.

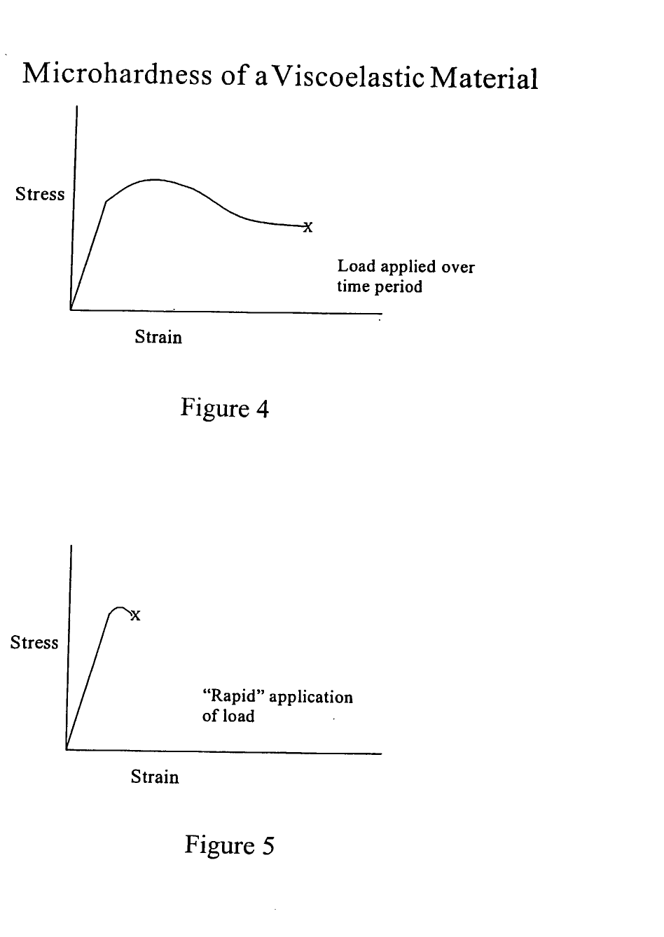

Bone is a viscoelastic material; thus, there is a function of time involved as bone undergoes deformation (FIG. 4). Currey and Brear (1990) found that when a load is applied for less than 10 seconds, the measured microhardness is greater than the microhardness measured when the load is applied for more than 10 seconds, which allows complete deformation of the bone sample. As a result of the “rapid” application of the load stress relaxation of the bone is reduced, and bone material properties become more ceramic in nature (FIG. 5).

Materials and Methods

Twenty-five maxillary hydroxylapatite implants (Interpore 200, Interpore International, Irvine Calif.) (4-138 months range, 32 month mean) were removed from 17 patients. These implants had been placed into the lateral maxillary wall during orthognathic surgery. The implants were harvested for analysis with voluntary consent from each patient in the process of performing other necessary surgery in the same area (i.e. removal of bone plates, sinus exploration, facial augmentation, etc.). Data from a subset of this group has been published elsewhere (Nunes et al. 1997). Pooling of data was possible because the same oral and maxillofacial surgeon performed the implantation and removal of the porous block HA grafts for each patient. Surgical procedures for removal of the specimens are described elsewhere, (Nunes et al. 1997).

Once the specimens were removed, they were fixed in 10% formaldehyde. They were then rinsed with phosphate buffered saline to remove the formalin in preparation for serial transverse sectioning to visualize consecutive interior surfaces of the implant. The implants were typically 7-10 mm. in length and 3-5 mm in thickness. The implant sections (and surrounding bone) were embedded in a nonpenetrating epoxy resin (Epo-Kwick, Buehler) to stabilize the specimens for machining. The samples were then sequentially sectioned in 1 mm increments using a 10.2 cm. diameter, 400 μm thick, diamond wafering blade and an Isomet Low Speed saw (Buehler). Six to nine sections, 10-25 mm² in area, were obtained for a typical implant (n. =1 to 4/patient).

Preparation of the implant samples for staining and subsequent histomorphometric measurements was done in a similar manner as described in Section B. Full color video capture of all the sections was obtained using far blue (visible, 405 nm wavelength) light to illuminate the samples. Images were stored on a computer disk to be directly analyzed at 1 12x final magnification to determine the percent of implant surface covered with bone and the percent of ingrowth into the pores within the implant.

Images were obtained from each implant cross section. The total area of the images accounted for more than 40% each implant's total cross-sectional area. Bone, void, and implant percentages were measured directly from the video images using point counting and SigmaScanPro software (Jandel Scientific). On each image, random points were counted using stereoscopic measuring techniques (Parfitt 1983) and the composition at each point was typed as either bone, void or implant. The ratio of these counts were used to calculate the histomorphmetric parameters of interest as described in Table IV.

Microhardness was measured using a Tukon MO microhardness tester with a 136° diamond pyramid indenter and a 50g load. Vicker's hardness number (VHN) was calculated from VHN=(2P sin(x/2))/d², where P is the applied load (g), x is the indenter angle (136°) and d is the mean of the two indent diagonals (μm). The length of d is on the order of 100 μm and is within the 230 μm diameter pore size. Care was taken to ensure the indent remained centered in the pore to reduce the potential for edge effects. Exterior bone was tested at a distance greater than 100 μm from the implant interface. Both bone and HA were tested in the implant interior (>300 μm from extant bone interface) and at the exterior bone-implant interface. The mean microhardness value was calculated from three microhardness measurements made from each of the areas.

Statistical testing was done using JMP statistical analysis software (SAS Institute, Inc.). A z-test was used to judge significance of implant composition and microhardness values. Single factor ANOVA was used to judge differences in microhardness values between measurement areas. All statistical tests were carried out at a level of α=0.05.

Results

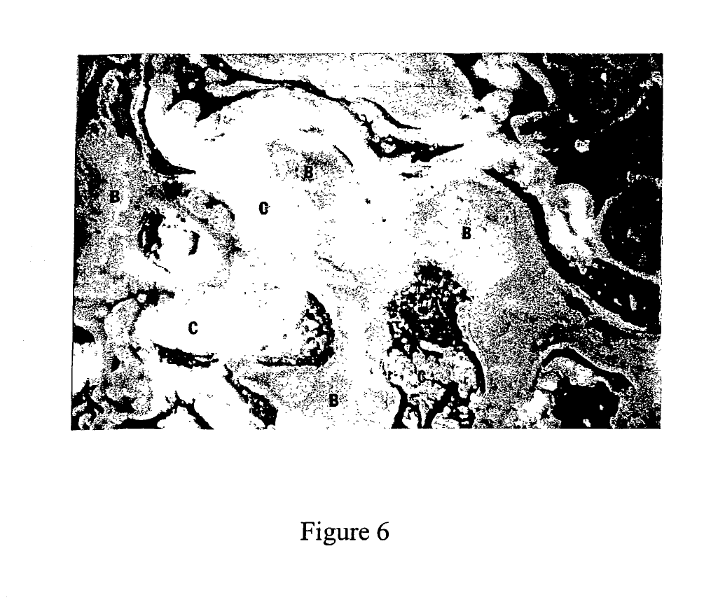

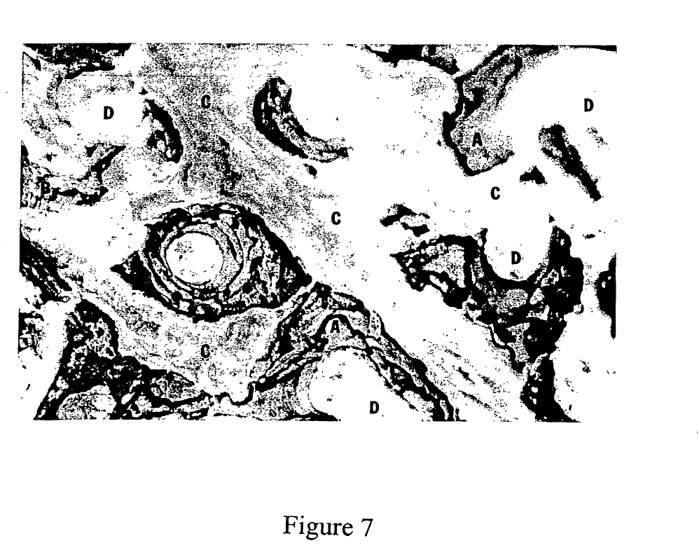

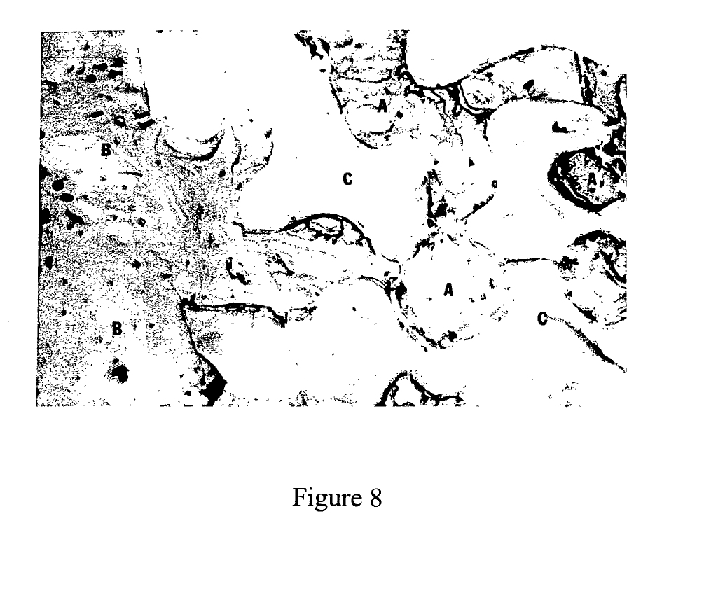

Microscopic analysis revealed microstructurally intact implants that were incorporated into the existing bone. The 4-month implant (FIG. 6) contained large amounts of soft tissue that was present in approximately three-quarters of the available pore space. Bone ingrowth was in the early stages, lining only the pore wall and penetrating into the implant no more than 300 μm from the interface. The bone in the implant interstices was of the woven type, with early stage lamellar type bone lining the pore walls. The later term implants (14-38 months) (FIG. 7) contained greater amounts of lamellar bone, which penetrated deeper (300-1000 μm) into the implant pores. Woven bone was still present in these implants, but in relatively reduced amounts. The oldest implants (>48 months) (FIG. 8) had Haversian-type bone present throughout the implant. No woven bone was discernible in these specimens and vascular and soft tissues filled the remaining pore space.

Active bone formation surfaces were noted in all of the specimens. These were marked by osteoblasts lying immediately against the osteoid seam, stained deep purple, with confluent granules visible against the mineralizing bone surface (Parfitt 1983). Specimens older than 14 months showed evidence of remodeling of the Haversian bone, with secondary cement lines clearly visible about the Haversian canals.

No macrophages (defined as mononuclear cells >40 μm) were visible in the pore spaces or adjacent to the implant surface. Because this study sought to characterize the material properties of the bone within the pores of the implant, a specific analysis of giant cell (polykaryon) adhesion was not carried out. Several previous studies have looked specifically at biocompatibility issues relating to porous block HA, including immune responses (Wolford et al. 1987; Jahn 1992; Nelson et al. 1993).

Sample composition and bone contact data are presented in Table VII.

TABLE VII
Porous Block Hydroxylapatite Implant Composition

| | | Sample Composition | | | % Ingrowth in | |
|---|---|---|---|---|---|---|
| | Duration | | Soft Tissue | | Available | % Apposition |
| Patient | (Months) | Bone | and Void | Implant | Space (Bone) | (Bone) |
| 1 | 4 | 0.05 | 0.36 | 0.59 | 0.12 | 0.13 |
| 2 | 14 | 0.26 | 0.34 | 0.41 | 0.43 | 0.48 |
| 3 | 14 | 0.27 | 0.16 | 0.58 | 0.63 | 0.61 |
| 4 | 14 | 0.19 | 0.33 | 0.48 | 0.37 | 0.52 |
| 5 | 14 | 0.17 | 0.40 | 0.43 | 0.30 | 0.45 |
| 6 | 16 | 0.33 | 0.12 | 0.56 | 0.73 | 0.74 |
| 7 | 16 | 0.19 | 0.23 | 0.57 | 0.45 | 0.45 |
| 8 | 18 | 0.26 | 0.10 | 0.64 | 0.72 | 0.77 |
| 9 | 18 | 0.19 | 0.31 | 0.50 | 0.38 | 0.55 |
| 1A | 19 | 0.18 | 0.31 | 0.51 | 0.37 | 0.58 |
| 10 | 20 | 0.28 | 0.28 | 0.44 | 0.50 | 0.53 |
| 11 | 21 | 0.28 | 0.25 | 0.47 | 0.53 | 0.60 |
| 12 | 23 | 0.25 | 0.12 | 0.63 | 0.68 | 0.69 |
| 13 | 30 | 0.29 | 0.27 | 0.44 | 0.52 | 0.57 |
| 14 | 38 | 0.21 | 0.34 | 0.46 | 0.38 | 0.52 |
| 16 | 128 | 0.27 | 0.32 | 0.42 | 0.47 | 0.57 |
| 17 | 138 | 0.31 | 0.22 | 0.47 | 0.59 | 0.55 |
| Mean | 32.06 | 0.23 | 0.26 | 0.51 | 0.48 | 0.55 |
| Std. | 38.72 | 0.07 | 0.09 | 0.07 | 0.16 | 0.14 |
| Dev. | | | | | | |

Overall mean composition of the implants was 23 ± 17% bone (range, 7-31%), 51 ± 7% HA matrix (range, 39-65%), and the remainder was soft tissue or void at 26 ± 9% (range, 10-40%). Percent ingrowth in available space (% IAS), defined as % bone/(% bone + % void), averaged 48 ± 16%.

The implant from the patient taken at 4-months post-surgery had 5% bone ingrowth. The second biopsy obtained from this patient, at 19 months, revealed bone ingrowth similar to that of implant biopsies of comparable duration (18%), with concomitant reduction in void space and HA.

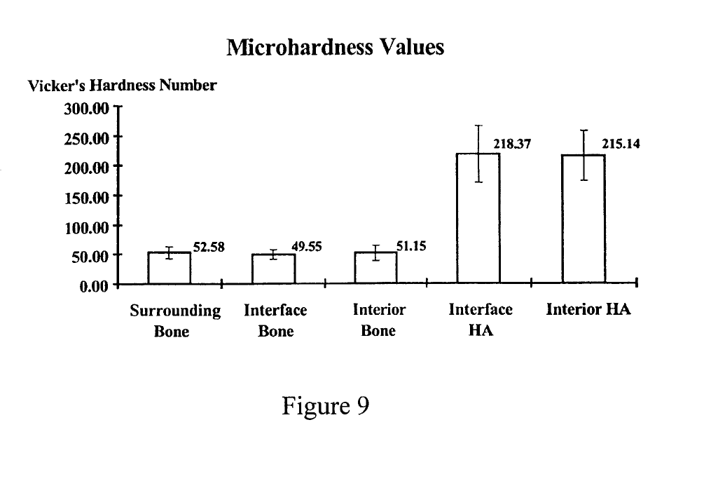

Microhardness measurements (FIG. 9) for bone and HA are in agreement with results from previous studies (Simske and Sachdeva 1995; Simske et al. 1995). Single factor ANOVA showed no significant differences in microhardness of the bone in the measured regions. There also were no significant differences between the interior and interface HA. There was insufficient osseous tissue in the pore spaces to allow microhardness testing of short-term. (<14 months in vivo). Thus the bone that was tested for microhardness was lamellar.

Discussion

This study is the first to examine porous coralline hydroxyapatite implants that have been in vivo for periods of up to 11.5 years. Using standard histomophometric techniques the amount of bone within the pore spaces and its surface contact with the implant was measured, allowing for the creation of an overall understanding of bone development and maturation during the “lifetime” of a porous maxillary implant.

Microscopic examination of the implant biopsies showed well incorporated porous block hydroxyapatite implants with viable soft tissue and bone present. Implants that had been in vivo for a short time contained primarily soft tissue within the pores. Tissue ossification was present in all samples with significant ingrowth, even with the shortest implantation. Bone growth within the pores was normal, with a layer of ostcobtasts forming an osteoid seam behind which mineralization was occurring.

Over time, the soft tissue gave way to primarily woven or lamellar bone. Bone within the long duration implants (138 and 128 months) was solely lamellar with significant Haversian systems. The Haversian systems had lamella and osteocytes located circumferentially around the Haversian canal, indicating that normal physiologic processes we re active in maintaining the bone within the implant (Parfitt 1983). The continued bone ingrowth over time, along with the lack of an apparent immune response and normal morphology of the bone surrounding the implants, indicated continued long-term biocompatibility of the porous block hydroxyapatite.

Histomorphometric measurements validate the histologic observations. Bone ingrowth and apposition was significant (23±7% and 55±14%, respectively, p<0.05). The amount of bone present in the implant increased with continued implantation time and appeared to begin to plateau around the 20 month time frame. It appears that the bone within an implant reaches an equilibrium in which the relative amount of osseous tissue remains constant. This has been observed in a previous study in which a near balance between the bone and implant was achieved (Nune et al. 1997). The reduction in time of the difference between apposition and ingrowth implies that as time increases, apposition reaches a final value and the bone fills in the pore space.

Consistent microhardness values, regardless of location (interior, interface, and surrounding), suggest that the material properties of the bone within the implants are equivalent to those of the surrounding bone. These values were within the range of similar osseous tissues measured previously (Simske and Saclideva 1995; Houde et al. 1995; Currey and Brear 1990; Simske et al. 1995). There was no discernible degradation of the porous block HA material, as exhibited by the consistent microhardness values in both the interior and at the interface. Because the bone that formed in the pores was similar to the surrounding bone, and the implant showed no significant degradation, the aggregate bone/porous block HA retained its structural integrity over very long periods aiding in the stability of the implant.

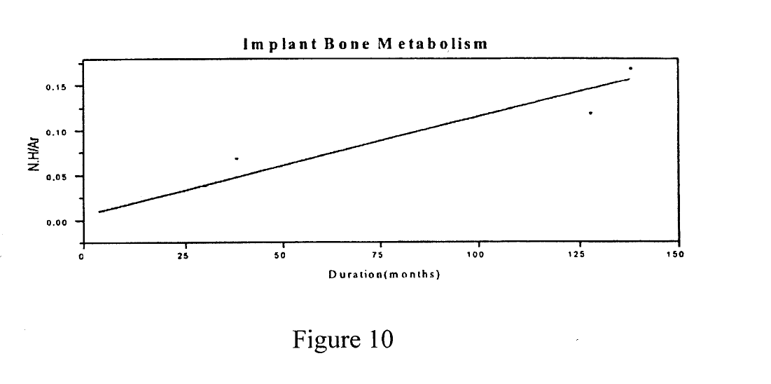

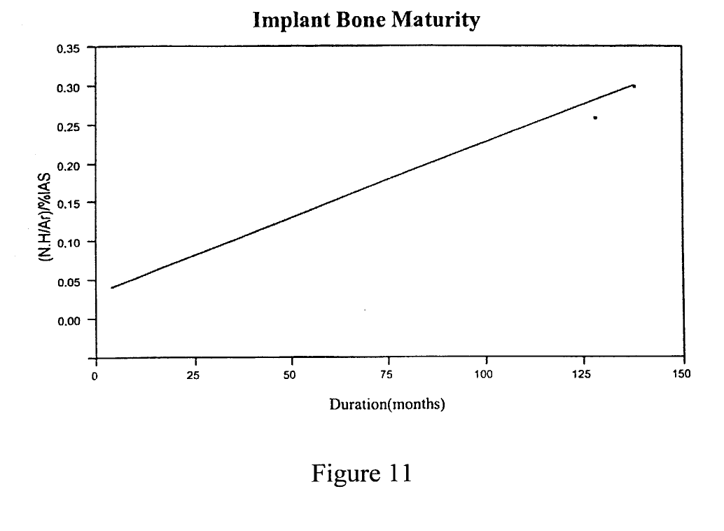

The number of Haversian systems present in bone can be considered an indicator of the metabolic activity in that local region (Parfit 1983). The bone metabolic activity for the transected surfaces imaged in this study was measured by counting the number of Haversian systems in the pores of the HA implants. There was a significant correlation between the number of Haversian systems per area of implant imaged (N.H/Ar) (Parfit et al. 1987) and the time of implantation (FIG. 10). When N.H/Ar is normalized with the percentage of bone that is actually in the pore spaces (N.H/(Ar*% IAS)) the correlation to implant duration remains significant (FIG. 11). Bone metabolic activity appears to increase as the time of implantation increases, signifying that, in general, porous block HA does not impede the normal metabolic processes of bone in the pores over long periods of time. The bone within the pores continues to mature over time so that only lamellar Haversian type bone is present. The continued maturation of the ingrown bone observed is consistent with a previous study that used chemical analysis to measure the maturity of the ingrown bone in Ti-6AI-4V porous fiber implants (Barth et al. 1986). Based on the findings in this study, porous block hydroxyapatite is a viable material for long term implantation to the maxilla in orthognathic surgery.

D. Quantification of Bone Ingrowth into Porous Block Hydroxyapatite in Humans

The work conducted in this section expands upon Section C by quantifying craniofacial bone ingrowth into porous implants both over the time in vivo (temporally) and depth into the implant from a known implant/extant bone interface (spatially). This is the first study of its type. Numerous hypotheses are examined here. Initial craniofacial bone ingrowth is primarily a function of the biologic action of the tissues (hypothesis 1B). Over time bone ingrowth asymptotically approaches a value with the relative amount of osseous tissue remaining constant (hypothesis 1D). Bone ingrowth decreases with increasing depth into the implant (hypothesis 1E); biologic factors are predominant at depths greater than 1 mm into the implant hypothesis 1F). Craniofacial bone ingrowth into porous implants can be systematically quantified temporally and spatially (hypothesis 2).

Seventeen maxillary hydroxyapatite implants (implant time of 4-138 months range, 39-month mean) were harvested for analysis from 14 patients. The implants had been placed into the lateral maxillary wall during orthognathic surgery, juxtapositioned to the maxillary sinus. Ingrowth was measured in 100 μm increments from a bone/implant interface to a depth of 1500 μm. Bone ingrowth, averaged over the 14 patients, from 0-1 100 μm depth, is described by the equation: % ingrowth=-20% *(depth in millimeters)+41.25% (R²=0.98, n=10 incremental depths). Beyond 1100 μm, the average ingrowth remained constant at 15.0+/−0.7%. Duration of implantation also showed an effect on the percent ingrowth into the implants at the incremental depths, with the percent ingrowth asymptotically approaching a maximum. Overall, the composite average data from all depths is best described by the logarithiic function % ingrowth =15% *Ln(Implantation Time in Months) -24.0% (R²=0.71, n=14 patients). Several factors may come into play in determining bone ingrowth including the mechanical environment, osteoconductivity of the implant material and osteogenic capability of the tissues in the pore spaces. Measurements of bone ingrowth are most influenced by the depth into the implant and the time the implant has been in the body, while the age of the patient has little effect on bone ingrowth.

Introduction

Porous alloplastic implants have been studied extensively for their use in oral and maxillofacial applications (Wolford et al. 1987; Holmes et al. 1988; Nunes et al. 1997; Ayers et al. 1998). The use of these materials allows for the cosmesis and continuity of the surrounding bony structures without the concerns associated with the use of autogenic implants. Other advantages of porous alloplastic implants in craniofacial applications include an increase in resistance to fatigue fracturing and greater resistance to separation (Eppley and Sadove 1990). Ceramic, porous block hydroxyapatite (HA), one such alloplastic implant, has been shown to be an effective implant material in both short and long term applications (Klinge et al. 1992; Jahn 1992; Nunes et al. 1997; Ayers et al. 1998).

The ingrowth of bone into porous materials is affected by the geometry and osteoconductivity of the substrate as well as time of implantation. It is understood that an implant must have a sufficient pore size (100-400 μm) for the development of mineralized bone (Klawitter and Hulbert 1971; Hulbert et al. 1970; Ripamonti 1991), along with interconnecting fenestrations between the larger pores to support the vascular tissue required for continued mineralized bone maturation (van Eeden and Ripamonti 1994; Eggli et al. 1988). Hydroxyapatite (HAP) has been shown to be osteoconductive, and in the form of the reef building coral, genus Porites (prepared commercially as Interpore 200®), it provides an appropriate structural scaffold upon which bone can grow (Martin et al. 1989). Bone ingrowth into the pore spaces of Interpore 200® has been shown to significantly increase over 20 months until an equilibrium condition, wherein bone ingrowth remains relatively constant, is obtained in the maxilla of humans (Ayers et al. 1998).

Previous work that has considered incremental bone ingrowth into a porous implant has focused primarily on the micro-mechanical environment of the bone/implant interface and the consideration that the stress transfer at the implant interface stimulates tissue differentiation (Prendergast et al. 1997). Local stress concentrations in bone diminish with increasing depths in a Sulemesh multilayer wire anchorage for the acetabulum, and the highest amount of stress transfer occurs in the first wire layer (300-500 μm) (Pedersen et al. 1991). In an analysis of ingrowth into a sintered porous bead structure, strain energy density data indicated that the final bone structure in and around a porous implant reflects both the loading and nutritional requirements of the bone (Hollister et al. 1993). In essence, bone ingrowth into the pore spaces does not create a structure optimized solely to the mechanical environment (Hollister et al. 1993). How ingrowth into porous implants in vivo reflects mechanical environment and simultaneous non-mechanical factors remains to be determined.

This study examines how bone ingrowth changes with depth into the implant from the interface (spatially), and with time of implantation in vivo (temporally). A preliminary examination of how patient age affects bone ingrowth is also provided. In so doing, important factors controlling in vivo bone ingrowth into porous implants used in oral maxillofacial applications can be elucidated.

Materials and Methods

Seventeen maxillary hydroxyapatite implants (implant time of 4-138 months range, 39 month mean) were removed from 14 patients (biopsies were obtained after 4, 14(n=3), 16, 18(n=2), 19, 30, 31, 38, 61, 128, and 138 months implantation). These constitute a subset of implants used in Section C, above. Implant placement and biopsy procedures are discussed in Section C, above. Fewer of the available implant biopsies were used in this work as only implants with a clear interface between surrounding extant bone and the implant were considered. Patient data pooling is possible as the same oral and maxillofacial surgeon performed the implantation and removal of the porous block HA grafts for each patient. Surgical procedures for the removal of the specimens are presented elsewhere (Nunes et al. 1997).

Preparation of the implant biopsies for analysis after their removal is presented in Section C. Staining techniques used for quantitative histomorphometric measurements are also presented in Section C.

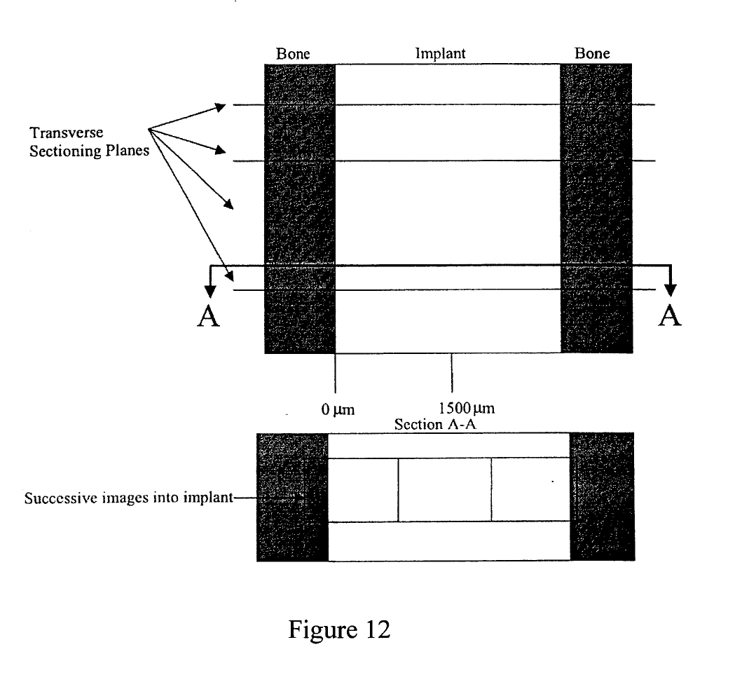

Full color digital imaging of all the sections was obtained using far blue (visible, 405 nm wavelength) light to illuminate the samples (Olympus AHBT-3 microscope, Olympus Inc., Tokyo, Japan with a CMOS-Pro digital camera, Sound Vision Inc., Framingham, Mass.). Each sequentially obtained image covered approximately 1 mm² in area (FIG. 12). The images were stored on computer disk to be directly analyzed at 330× final magnification to determine the percent of ingrowth into pores throughout the implant.

Quantitative measurements were made sequentially over 100 μm increments (e.g. 0-100 μm, 100-200 μm, etc.) through the cross-section to a depth of 1500 μm from the exposed surfaces of each implant. Bone, void, and implant percentages were measured directly from the video images using point counting and SigmaScanPro software (Jandel, San Rafael Calif.). A unique application of these measurements was accomplished by starting from the interface between surrounding bone and the implarit and counting random points over each 100 μm increment using stereoscopic measuring techniques (Parfitt, 1983) and each point was classified either bone (A_{B}) void (A_{V}) or implant (A_{I}). The 1500 μm depth from a known implant/extant bone interface ensured that there were no edge ingrowth effects from other faces of the implant (i.e. all implants were at least 3 mm a side in dimension). Successive cross-sections from each implant were then combined to obtain the final point count for each implant (2000-3500 total points per implant). The total points in each category were then divided by the total number of points measured (Aₜ) to obtain % bone (A_{b}/Aₜ), % void and soft tissue (Aᵥ/Aₜ), and % implant (A_{I}/Aₜ). The % ingrowth into available space within the implant was defined as % bone/(% bone+% void) and is the area ratio of ingrown bone to total non-implant area.

Applying principles used in signal processing, ingrowth values at the sequential depths were obtained using 300 μm moving averages (e.g. the average ingrowth at the 100-200 μm increment is the average of the values taken from the 0-100, 100-200 and 200-300 μm increments). This method accounts for slight discontinuities caused by a relatively large pore size (230 μm) compared to step size (100 μm). The values of each implant were then examined as a function of depth into the implant or as a function of implantation time at a specific depth.

Curve fitting routines were then applied to describe the asymptotic nature of percent ingrowth with respect to depth into the implant and percent ingrowth with respect to the time of implantation (JMP, SAS Institute, Inc.). The curve fit used for the % ingrowth as a function of depth into the implant was piecewise linear. The asymptotic nature of the data describing % ingrowth with respect to the time of implantation at each incremental depth suggested a logarithmic function (% ingrowth=Ln (implantation time)+b) where b corresponds to the y-intercept. Correlation coefficients were determined in order to verify the significance of the fitted curves given the number of data samples. Statistical testing was done using JMP statistical analysis software (SAS Institute, Inc.). All statistical tests were carried out at a level of α=0.05).

Results

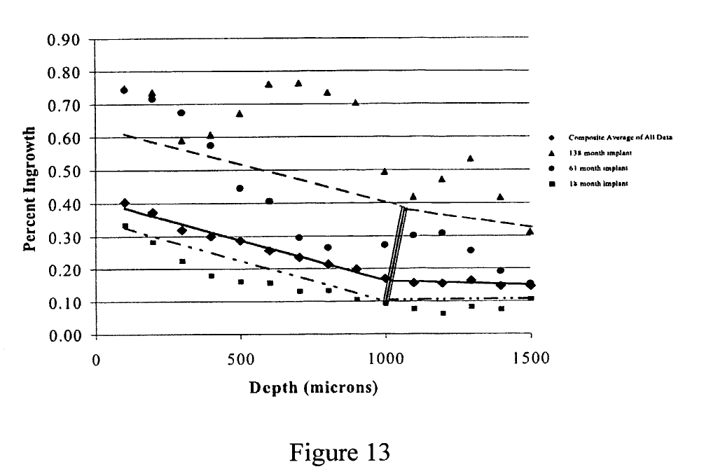

All implants, other than the implant taken at 128 months, displayed decreasing percentage ingrowth with increasing distance into the implant interior from the interface. The implant that did not show a decreased ingrowth with depth had constant ingrowth of 38+/−5% throughout the implant cross-section. Combining all implant data to create a composite average allowed for an examination of bone ingrowth without regard to the time of implantation of the HA. Composite average bone ingrowth decreased rapidly in a linear fashion starting at 40% ingrowth from the 0-100 μm increment from the implant interface and decreasing to 15% at the 1000-1100 μm increment in the interior. Ingrowth was constant at approximately 15% beyond 1100 μm (FIG. 13). Individual patient data mirrored this trend at higher or lower ingrowth values, depending upon the time of implantation, The percentage of ingrowth (independent of time of implantation), given as a function of the depth into the implant, appears to be most simply described as a piecewise linear relationship. The decrease in ingrowth for the 14 patients across implant depths of 0-1100 μm is described by the composite average equation: % ingrowth=−20% *(depth in millimeters)+41.25% (R²=0.98, n=10 100 μm increments). Beyond 1100 μm, % ingrowth remains constant at 15.0+/−0.7% (R²=0.34).

The piecewise linear relationship exemplified by the composite average is influenced by the time of implantation. The absolute ingrowth values of the curves are shifted up for the longer-term implants (>48 months) and down for the shorter-term (<48 months) implants. Implants with implantation duration over 48 months (3 patients) had an average linear decrease of ingrowth described by the equation: % ingrowth=−20% *(depth in millimeters)+63.36% (R²=0.90). Implants with implantation duration under 48 months (3 patients) had an average linear equation of % ingrowth =-30% *(depth in millimeters)+35.22% (R²=0.96). Interior ingrowth also increased or decreased proportionally depending upon implantation time yet remained relatively constant with respect to the measurements taken beyond the first 1000 μm into the implant (1 100-1500 μm ).

Pore size did not appear to have an effect on the ingrowth. Because of the structural nature of the Porites coral, it has roughly a bimodal pore size (albeit not statistically significant), with the larger pores averaging 230 μm and the interconnecting fenestrations averaging 190 μm. It was noted that regardless of the sectioning plane (e.g. perpendicular to the large pores or small pores) the % ingrowth did not change with respect to the surfaces imaged.

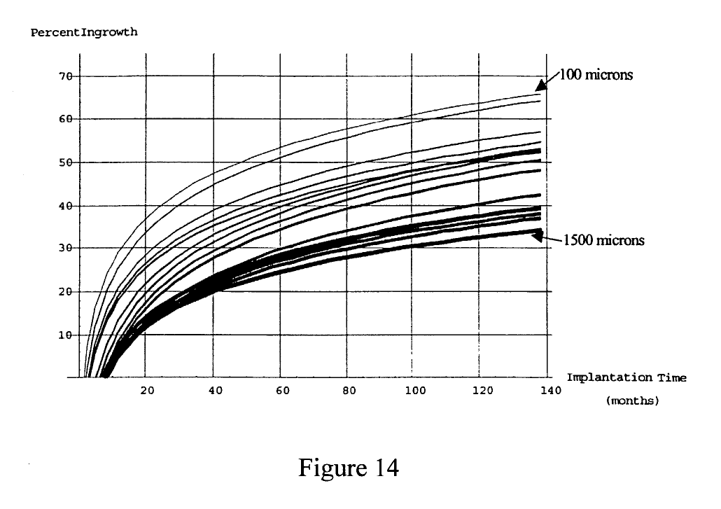

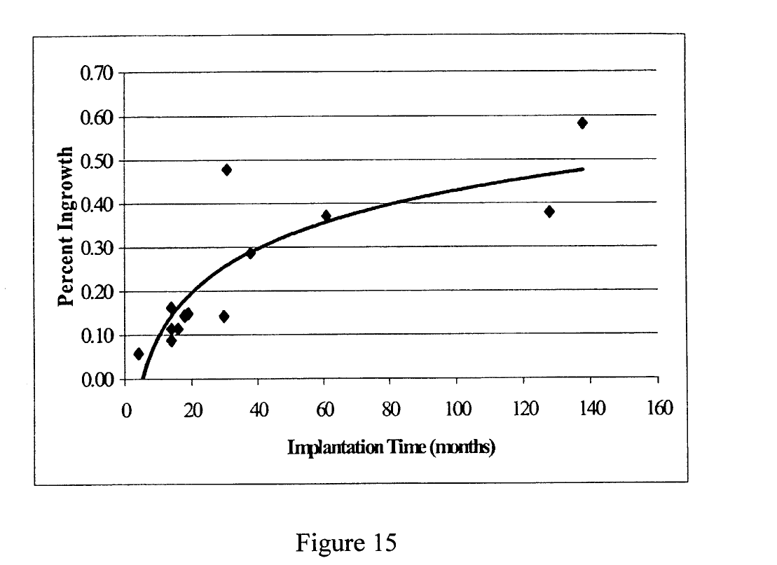

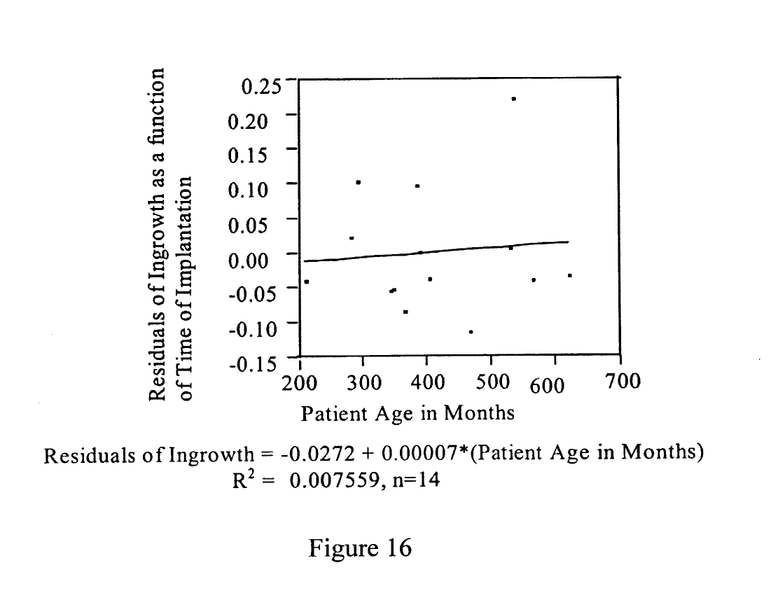

Time of implantation showed an effect on the % ingrowth into the implants at the implants at the incremental depths. At each depth the % ingrowth asymptotically approaches a maximum that fits a logarithmic curve (FIG. 14). The curve at each specific depth is similar to the curves at the other depths. Thus a family of curves describing ingrowth as a function of implantation duration is obtained. Overall, the combined data from all depths is best described by the logarithimic function % ingrowth 15%*Ln(Implantation Time in Months)−24% (R² 0.71, n=14) (FIG. 15). A comparison of the residuals from the percent ingrowth as a function of the time of implantation indicates patient age (range 17-52 years, mean 34 years 3 months) had no significant effect upon bone ingrowth at specific depths (FIG. 16).

Discussion

In this study, bone ingrowth into the spaces of porous block HA used in maxillofacial applications is quantified both temporally and spatially. The progression of bone into the implant appears somewhat nonlinear (fluctuating) when looking at a single sample: there is a large influx of bone (seen in the deeper regions of the pore spaces) followed by a slight reduction and then an another increase. This is best exemplified by the individual samples taken at 61 and 128 months (FIG. 13). The cause of this phenomenon is unknown, but is likely indicative of a continual process of bone turnover within the implants, even after as much as ten years time.

Bone ingrowth as a function of the depth into the implant is most simply shown using a piecewise linear model where ingrowth in the first 1000 μm follows, on average, a linear reduction from 40% ingrowth to 15% ingrowth and is best described by the equation: % ingrowth =-20%*(depth in millimeters)+41.25% (R²=0.98, n=10 100 μm increments). Other curve fits applied to this data did not yield significant increases in R² values. The time of implantation affects this curve. Longer-term implants had higher percentages of bone ingrowth, but the rate of reduction of ingrowth as depth into the implant increased did not appear to significantly change from the overall average described previously. There is perhaps a shift in the rate of reduction, but the low number of older implants prevented an accurate measurement of this. Short-term implants displayed lower ingrowth, yet still followed approximately the same rate of reduction in bone ingrowth described by the linear fit.

These findings suggest that a combination of factors are at work in affecting bone ingrowth into porous materials. The reduction of bone ingrowth over the first 1000 μm may be primarily due to reduced load transmission from the implant to the bone. The stresses and strains required to encourage bone growth are reduced at the deeper regions of the implant due to load transfer occurring predominantly through the bone ingrown at the outermost regions of a porous coating (Pedersen et al. 1991). The constant value of ingrowth at the deeper regions of the implant, where it may be expected that stress shielding would be at a maximum, may be a function of the osteoconductivity of the HA and the osteogenic capacity of the tissues present in the pore spaces (Chang et al., 1996). As such, different bone ingrowth values for different implant materials are expected, dependent on the relative osteoinductive and osteoconductive capabilities of the materials. The reduction of bone ingrowth with increasing depth into the porous HA is similar to previously reported results, where it was noted that the percentage of bone at given depths declined considerably from the surface to the center of 3 mm diameter cylinders implanted into the cancellous bone of rabbits (Eggli et al. 1988).

The point at which bone ingrowth changes to a constant value shifts towards the interior of the implant as time increases. This is noted by the highly sloped triple line on FIG. 13. At this point the bone should be entirely stress-shielded by the implant. In these particular implants, bone ingrowth remains constant after that point. Thus, this line may be considered a demarcation where mechanical influences on bone ingrowth are exceeded by the biological influences.

Previous work has also shown that bone ingrowth continues over the course of time (Oberg and Rsenquist 1994; Martin et al. 1993; Hofmann et al. 1997). Martin et al. (1993) demonstrated that ingrowth into Interpore 200® after 1 year reached a level of 74% when placed in the cortical bone of the radius of dogs. Hofmann et al. (1997) found that bone ingrowth into porous titanium implants used in human total knee arthroplasty plateaued in approximately 9 months at a value of 24%. These results indicate similar asymptotic trends to what is herein reported when bone ingrowth is measured over time at specific depth into the implant. The logarithmic nature of each of the curves derived here suggest that bone ingrowth ateach depth will reach a saturation value that is specific to the depth. The fact that these curves predict little or no bone ingrowth at the interface in the first month is borne out by an implant biopsy taken at 3 weeks, which had little measurable ingrowth even at the interface between existing bone and the implant. An implant biopsy taken at 4 months, however, displayed measurable ingrowth. It was also noted that little significant bone ingrowth appeared in the interior regions of the implants until 14 months, again matching the predictions given by the logarithmic curves. Overall, there appears to be a maximum ingrowth at each depth in the implant. These maxima decrease with increasing depth into the implant. Thus, it may be expected that over a sufficient time period, bone ingrowth will reach an equilibrium condition at all depths into a porous implant. This equilibrium value may be affected by the specific implant material chosen, although this was not addressed herein.

Although it was not examined here, the measurements of bone ingrowth may be affected by the accessibility of the pore spaces to vascularized tissue, especially in the shorter-term (<48 month) implants. Such an effect is unlikely, however, to be the mechanism underlying the curves obtained for the long-term implants. In these older implants, the pore spaces are filled with Haversian type bone, which would suggest that the vascularization of the pore space is complete and mature bone is present. In these long-term implants, decreasing amounts of bone ingrowth are still observed as the depth into the implant increases, even though the porosity in the interior regions is equivalent to that at the surface.

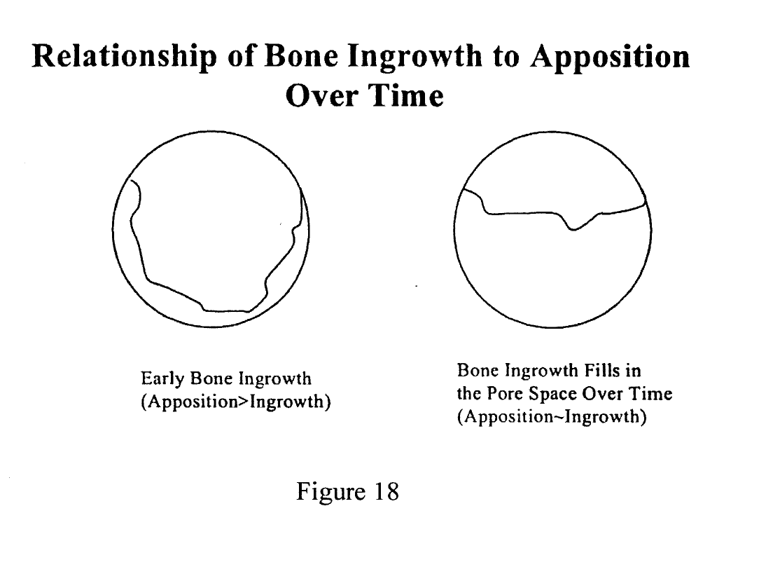

Within the pore spaces of the implants, % apposition against the HA was observed to be greater than % ingrowth into the available space for all lengths of implantation time. The percent difference between these two measures was, in general, higher in the shorter-term implants (22% for implantation times <48 months) than in longer-term implants (6% for implantation times >48 months). The significance of this is unknown, but it may indicate an ongoing turnover of bone within the implants or that apposition remains relatively constant after a period of time while bone ingrowth “fills in” or matures within the pore space (FIG. 18).

A previous study noted that bone ingrowth appeared to be “held up” at the interface of 30% (230 μm mean pore size) porous HA while a 50% porous (178 μm mean pore size) nitinol specimen had greater ingrowth (Simske and Sachdeva, 1995). This highlights the relationship between the measurement of bone ingrowth and pore size, If one was to consider two pores (one large than the other) in similar material, with equivalent amounts of bone present. As can be readily seen, the smaller pore will have high ingrowth and apposition measurements, while the larger pore will have low ingrowth but high apposition. If two dissimilar materials were used (e.g. HA and nitinol) the ingrowth and apposition measurements would be affected by the osteoconductive nature of the HA and ostopermissive nature of the nitinol. With a material that is osseoconductive such as HA, the cellular differentiation near the surface would encourage increased apposition of the bone over the ingrowth. If the material such as nitinol is used, apposition may remain similar in value to ingrowth as there is no osteoconductive or osteoinductive influence on the mesenchymal cells near the nitinol surface. What may be established from the discussion on material selection and bone ingrowth measurements is that one must consider the many factors that affect bone ingrowth into porous materials when comparing different materials and porosities.

While previous work has shown that bone ingrowth into a porous implant is not optimized nor predicted exclusively by the loading environment (Hollister et al. 1993), this study has shown that bone ingrowth follows a predictable progression into the pore spaces of an implant or coating when used in maxillofacial applications. Ingrowth measurements were most affected by the depth into the implant and time of implantation. Patient age had very little effect on the amount or time for bone to grow into the available pore spaces. Further work must be done to fully elucidate the effect of pore size and density on these measurements, but this does present a unique capability in future porous implant design.

### Conclusions and Future Work

From the discussion presented above in Section A one may conclude that there is, most likely, no one optimum porous material for use in craniofacial applications. If one were to establish minimum requirements for the success of an implant, one may suggest that the implant must be biocompatible, osteopermissive at worst, but preferentially ostoconductive and osteoinductive. The mechanical properties (both as a solid material and as a porous device) of such an implant would allow for an even transfer of load between the surrounding bone and the implant to reduce the effects of stress- shielding. While a bioresorbable implant may be preferred, there exist sufficient number bone diseases and conditions that require that a permanent implant must be used (e.g. joint replacement in osteoporosis patients).

What is done in this dissertation is not to define the optimum material or porosity for bone ingrowth, but rather to establish a baseline of how craniofacial bone interacts with porous biomaterials over time. This allows one to understand what is required to optimize porous implant design for the greatest desired effect. As a rough overview of the work done herein, Section B indicated that the biological functions for bone growth (i.e. fracture healing) in the first 6-weeks following implantation have greater effect than porosit. However, in Section C, it becomes apparent that other factors affect craniofacial bone ingrowth in longer implantation times as indicated by the plateau of bone ingrowth occurring around 20 months. In Section D it is determined that these factors include, but may not be limited to, time of implantation, depth into the implant, and porosity of the implant.

Section B sought to elucidate the effect of pore size on craniofacial bone ingrowth for a specific material. This is similar to work done by Klawitter and Hulbert (1971) in which they implanted porous Ceroseium ceramic in the femurs of adult dogs. In the work conducted here, however, a porous metal was used in the cranial bones of rabbits. This is the first study to attempt to quantify the effect of porosity control on cranial bone ingrowth. It was shown that pore size has no significant affect on bone ingrowth during the early phases of bone ingrowth. This ingrowth was marked by the presence of woven bone within the pore spaces in all implant samples. Thus, it may be concluded that during the initial bone ingrowth into an implant, the mechanism for ingrowth is not highly dependent upon the pore size, as proposed in HypothesislA.

A likely explanation may be that the 6-week time period is still within the cellular response phase (i.e. fracture healing) (Shacowitz,). Bone formation at this point is primarily a function of the differentiation of mesenchymal tissue present in the pore spaces into ostcoblasts. The bone formed during this phase is Woven bone. This was noted in this study and provides a starting point to examine hypothesis 1B. It may be argued in this study, that the osteogenic capacity of the infiltrating tissue and the biologic functions of bone ingrowth are predominant during the early phase of bone ingrowth in implants with porosities within the established range of porosities required for long-term bone ingrowth (100-400 mm).

Section C begins to examine the effect of time of implantation on craniofacial bone ingrowth and apposition in a clinically accepted orthopedic biomaterial. This study was the first to look at porous implant biopsies that had been in vivo for more than a decade, separately or in addition to implant biopsies taken as early as 4 months in vivo. This section also elucidated the effect of time on bone ingrowth and apposition, establishing that bone ingrowth asymptotically reaches a consistent value circa 20-months post implantation (Hypothesis ID). Section C also establishes, through microhardness measurements, that mature lamellar ingrown craniofacial bone is similar in material properties to surrounding extant bone and that the material integrity of the HA does not appreciably degrade even after 11.5 years post implantation. This finding, in conjunction with histologic examination establish that craniofacial bone continues to mature within the pore spaces over extended periods of time (Hypothesis IC).

Section D builds upon Section C by specifically identifying where craniofacial bone ingrowth occurs, from a given implant/extant bone interface, and when this bone ingrowth occurs over a time of implantation ranging from 4 to 138 months. This was the first time that a systematic measure of craniofacial bone ingrowth into an implant over time has been conducted. This study does point out some interesting implications in porous implant design when considered for use in craniofacial bone.

The finding of a piecewise linear relationship between bone ingrowth and depth into the implant point towards a balance between the mechanical loads imparted on the bone and implant and the biological baseline for bone ingrowth into the pore spaces. Craniofacial bone ingrowth decreased with increasing depth into the implant most likely as a result of stress shielding (i.e. Wolff's Law). However, significant bone ingrowth was still observed in the interior spaces. The constant nature of this “deep” ingrowth implies that biologic aspects of craniofacial bone ingrowth are predominant at depths over Irrim into the implant (Hypothesis IF). This finding gives rise to the possibility that a functionally graded porosity may be a more efficient use of the pore spaces, balancing the space available for tissue ingrowth and the surface area for mechanical load transfer.

Ingrowth of bone into a porous implant over time follows a logarithmic function that is specific to each depth and indicates that the percent ingrowth of bone will reach an asymptote specific to the depth into the implant. This saturation value, at which the relative amount of osseous tissue at each depth remains constant, again indicates that functionally graded porosities may be used to optimize the pore space for craniofacial bone ingrowth. Although not tested in this work, the level bone ingrowth at the interface between the implant and surrounding bone may be such that the surface porosity of the aggregate implant/ingrown bone may approximate the porosity of extant bone (i.e. the resultant porous implant/ingrown bone aggregate will be 30% porous at the surface). Results of Section D also indicate that facial bone is programmed for growth as exhibited by the lack of correlation of patient age with bone ingrowth.

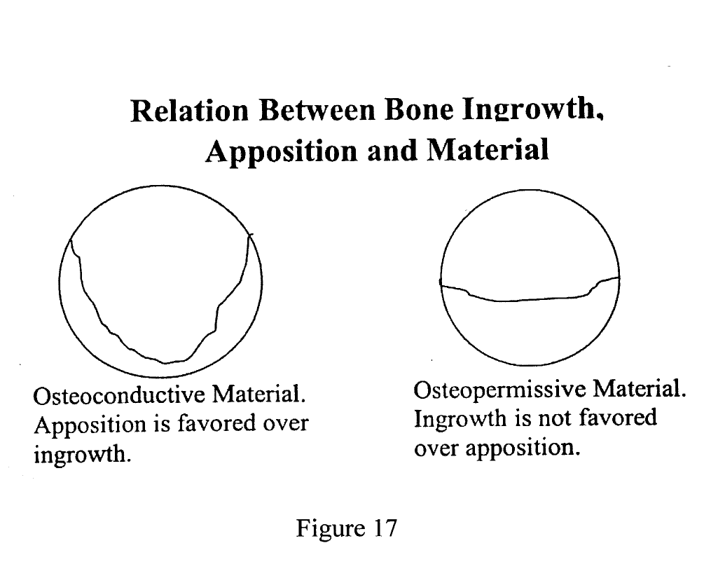

Differences between apposition and bone ingrowth measurements from all three sections above provide insight to the process of craniofacial bone ingrowth into the pore spaces. Apposition remained greater than bone ingrowth for all implantation times. This relative difference may be a possible indicator of the osteoconductivity of the material used in the bone or the time of implantation for a particular material. These potential relationships are shown in FIGS. 17 and 18. An osteoconductive/osteoinductive material will encourage cellular differentiation and attachment, or apposition, to the material surface over ingrowth resulting in a large difference in apposition and ingrowth measurements. An osteopermissive material, which is inert to the body, will not encourage apposition over ingrowth and hence the differences between the two measurements will be smaller than that of a bioactive material.

The difference in apposition and ingrowth measurements may also serve as an indicator of the time of implantation for a given orthopedic biomaterial. This relationship was noted in Section D and is best described by FIG. 18.

The present invention demonstrates that porous block HA has been shown to be an effective implant material over very long implantation times. Porous NiTi does appear to be sufficiently versatile as a material to warrant its consideration in bone engineering. The potential for modification of NiTi's surface properties to create a bioactive implant, similar to porous block HA, is further encouragement.

Much work has yet to be done to fully characterize porous NiTi as a material for bone engineering. This work ranges from refining the formation and processing of NiTi to rendering NiTi bioactive. In the area of materials processing, it has been demonstrated that ceramics can be combined with NiTi to create a composite or aggregate material (Itin et al. 1997). The incorporation of a superelastic shape-memory alloy enhances the tensile strength properties of the ceramic, while the ceramic provides the bioactivity for increased ingrowth of tissue (Itin et al. 1997). It is very feasible that a NiTi core with a bioactive ceramic outer surface can be created using SHS. There would be no interface between the ceramic and NiTi, as the transition from one to the other would occur over a functional gradient. In so doing the material and mechanical properties of the surrounding bone are matched with the ceramic, providing a bioactive surface for osseointegration, reducing the time for mineralized tissue infiltration and consequently patient recovery time.

Self-propagating-high-temperature-synthesis (SHS) has been demonstrated as an effective method of manufacture of porous metals, glasses and ceramics. Using this production method to create porous nitinol and HA based ceramics one can further to quantify the nature of bone ingrowth into porous orthopedic biomaterials used in other applications in vivo. The methodologies used here to quantity the bone in craniofacial bone implants could easily be adapted to quantify this relationship. SHS would then allow for the creation of specific implant morphologies to examine this and other questions.

The present invention may lead to creation of a more efficient implant interface with functionally graded porosities, where the surface pore size is sufficient to allow for a rapid influx of tissue and scales down towards the center of the implant. Depending on the implant application, the interior could remain solid for implants subjected to high loading environments, or be porous, allowing for vascular tissue ingrowth and later bone maturation.

Cytokine infiltration of implants is the addition of bone affecting proteins into the pore spaces of the implant, and offers the opportunity to improve the initial fixation at the bone/implant interface by enhancing the early ingrowth phase. Reagent infiltration of porous NiTi has not yet specifically been examined. However, bone morphogenic protein infiltration of porous materials is currently being examined using porous B₄C+Al₂O₃ and commercially pure titanium, reinforced with titanium boride, created with SHS and infiltrated with a bovine derived Bone Protein (Sulzer Orthopedics Biologics, WheatRidge, Colo.) in a rat skull on-lay model. Histologic analysis, bone ingrowth and surface contact measurements are currently being conducted. Implantation of reagent infiltrated SHS produced porous NiTi and bioglasses using the same methods is also underway.

The foregoing description is considered as illustrative only of the principles of the invention. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily will readily occur to those skilled in the art, it is not desired to limit the invention to the exact construction and process shown described above. Accordingly, all suitable modifications and equivalents may be resorted to falling within the scope of the invention as defined by the claims which follow.

### REFERENCES

1. Agrawal C M, Best J, Heckman J D, Boyan B D (1995) Protein release kinetics of a biodegradable implant for fracture non-unions. Biomaterials 18:1255-1280.

2. Airoldi G, Riva G (1996) Innovative materials: the NiTi alloys in orthodontics. Biomed Mater Eng 6:299-305.

3. Ayers R A, Simske S J, Bateman T A, Petkus A, Sachdeva RLC, Gyunter VE (1999) Effect of nitinol implant, porosity on cranial bone ingrowth and apposition after 6 weeks. J Biomed Mater Res 45:42-47.

4. Ayers R-A, Simske S J, Nunes C R, Wolford L M (1998) Long-term bone ingrowth and residual microhardness of porous block hydroxyapatite in humans. J Oral Maxillofac Surg, 56:1297-1301.

5. Ayers R A, Wolford L M, Bateman T A, Ferguson V L, Simske S J (in press) Quantification of bone ingrowth into porous block hydroxyapatite in humans. J Biomed Mater Res

6. Bajpai P K, Billotte W G (1995) Ceramic biomaterials. In: Bronzino J D (ed) The biomedical engineering handbook, CRC Press, Boca Raton, p 552.

7. Berger-Gorbet M, Broxup B, Rivard C, Yahia L H (1996) Biocompatibility testing of NiTi screws using immunohistochemestry on section containing metallic implants. J Biomed Mater Res 32:243-248.

8. Bragdon C R, Burke D, Lowenstein J D, O'Connor D O, Ramamurti B, Jasty M, Harris W H (1996) Differences in stiffness of the interface between a cemetless porous implant and cancellous bone in vivo in dogs due to varying amounts of implant motion. J Arthroplasty 11:945-951.

9. Broz J J, Simske S J, Corley W D, Greenberg A R (1997) Effects of deproteinization and ashing on site-specific properties of cortical bone. J Mater Sci Mater Med 8:395401.

10. Castleman L S, Motzkin S M, Alicandri F P, Bonawit V L (1976) Biocompatibility of nitinol alloy as an implant material. J Biomed Mater Res 10:695-731.

11. Dai K (1996) Studies and applications of NiTi shape memory alloys in the medical field in China. Biomed Mater Eng 6:233-240.

12. Desilets C P, Marden L J, Patterson A L, Hollinger J O (1992) Development of synthetic materials for craniofacial reconstruction. J Craniofac Surg 1: 150-153.

13. Ducheyne P (1998) Bioactive calcium phosphate ceramics and glasses. In: Sedel L, Cabanela M E (eds) Hip surgery: Materials and developments, Mosby, St. Louis, p 75.

14. Eggli P S, Muller W, Schenk R K (1988) Porous hydroxyapatite and tricalcium phosphate cylinders with two different pore size ranges implanted in the cancellous bone of rabbits. A comparative histomorphometric and histologic study of bony ingrowth and implant substitution. Clin Orthop 232:127-138.

15. Endo K (1995) Chemical modification of metallic implant surfaces with biofunctional proteins (part 1) molecular structure and biological activity of a modified NiTi alloy surface. J Dent Mater 14:185-198.

16. Endo K (1995) Chemical modification of metallic implant surfaces with biofunctional proteins (part 2) corrosion resistance of a chemically modified NiTi alloy. J Dent Mater 14:199-210.

17. Engh C A, Bugbee W D (1998) Extensively porous-coated femoral stems. In: Sedel L, Cabanela M E (eds) Hip surgery: Materials and developments, Mosby, St. Louis, p 243.

18. Eppley B L, Sadove A M, (1990) Effects of material porosity on implant bonding strength in a craniofacial model. J Craniofac Surg 1: 191-195.

19. Gao T J, Lindholm T S, Kommonen B, Ragni P, Paronzini A, Lindholm T C, Jalovaara P, Urist M R (1997) The use of a coral composite implant containing bone morphogenetic protein to repair a segmental tibial defect in sheep. Int Orthoped 21:194-200.

20. Greene D, Pruitt L, Maas C S (1.997) Biomechanical effects of e-PTFE implant structure on soft tissue implantation stability: a study in the porcine model. Laryngoscope 107:957-962.

21. Guicheux J, Gauthier 0, Aguado E, Heymann D, Pilet P, Couillaud S, Faivre A, Daculsi G (1998) Growth hormone-loaded marcroporous calcium phosphate ceramic: in vitro biopharmaceutical characterization and preliminary in vivo study. J Biomed Mater Res 24:47-63.

22. Hollinger J (1993) Strategies for regenerating bone of the craniofacial complex. Bone 14:575-580.

23. Hollister S J, Kikuchi N, Goldstein S A (1993) Do bone ingrowth processes produce a globally optimized structure? J Biomech 26:391-407.

24. Holmes R E, Wardrop R W, Wolford L M (198 8) Hydroxylapatite as bone graft substitute in orthograthic surgery: histologic and histometric findings. J Oral Maxillofac Surg 46:661-671.

25. Hulbert S F, Young F A, Mathews R S, Klawitter J J, Talbert C D Stelling F H (1970) Potential of ceramic materials as permanently implantable skeltal prostheses. J Biomed Mater Res 4:433-456.

26. Itin V I, Gyunter V E, Shabalovskaya S A, Sachdeva RLC (1994) Mechanical properties and shape memory of porous nitinol. Materials Characterization 32:179187.

27. Itin V I, Shevchenko N A, Korosteleva, Tukhfatullin A A, Mirgazizov M Z, Gyunter V E (1997) “Bioceramic-titanitim nickelide” functional composites for medicine. Tech Phys Lett 23:294-296, 1997.

28. Jahn A F (1992) Experimental applications of porous (coralline) hydroxylapatite in middle ear and mastoid reconstruction. Laryngoscope 102:289-299.

29. Kent J N, Zide M F (1984) Wound healing: bone and biomaterials. Otolarynogol Clin North Am 17:273-319.

30. Kieswetter K, Schwartz Z, Dean D D, Boyan B D (1996) The role of implant surface characteristics in the healing of bone. Crit Rev Oral Biol Med 7:329-345.

31. Klawitter J J, Hulbert S F (1971) Application of porous ceramics for the attachment of load bearing internal orthopedic applications. J Biomed Mater Res Symp 2:161-229.

32. Lakes R (1995) Composite biomaterials. In: Bronzino J D (ed) The biomedical engineering handbook, CRC Press, Boca Raton, p 598.

33. Light M, Kanat 10 (1991) The possible use of coralline hydroxyapatite, as a bone implant. J Foot Surg 30:472-476.

34. Martin R B I, Chapman M W, Sharkey N A, Zissimos A G, Bay B, Shors EC (1993) Bone ingrowth and mechanical properties of coralline hydroxyapatite 1 yr after implantation. Biomaterials 14:341-348.

35. Meng-Hai B, Xing-Yan L, Bao-Feng G, Chao Y, Dong-An C (1996) An implant of a composite of bovine bone morphogenetic protein and plaster of paris for treatment of femoral shaft nonunions. Int Surg 81:390-392.

36. Motoki I D S, Mulliken J B (1990) The healing of bone and cartilage. Clin Plast Surg, 17:527-544.

37. Neo M, Voigt C F, Herbst H, Gross U M (1998) Osteoblast activity at the interface between surface-active materials and bone in vivo: a study using in situ hybridization. J Biomed Mater Res 3 9: 1-8.

38. Nunes C R, Simske S J, Sachdeva R, Wolford L M (1997) Long-term ingrowth and apposition of porous hydroxylapatite, implants. J Biomed Mater Res 36:560-563:

39. Ono K, Yamamuro, T, Nakamura T, Kokubo T (1990) Mechanical properties of bone after implantation of apatite-wollastonite, containing glass ceramic-fibrin mixture. J Biomed Mater Res 24:47-63.

40. Otsuka K, Wayman C M. 199 8) Mechanism of shape memory effect and superelasticity. In: Otsuka K, Wayman C M (eds) Shape memory materials, Cambridge University Press, Cambridge, p 27.

41. Pederson D R, Brown, T D, Brand R A. Interstial bone stress distributions accompanying ingrowth of a screen-like prosthesis anchorage layer. J Biomech 24.

42. Phillips J H, Forrest C R, Gruss J S (1992) Current concepts in the use of bone grafts in facial fractures. Clin Plast Surg 19:41-58.

43. Putters J L M, Kaulesar Sukul DMKS, deZeeuw G R, Bijma A, Besselink Pa (1992) Comparative cell culture effects of shape memory metal (Nitinol), nickel and titanium: A biocompatibility estimation. Eur Surg Res 24:378-382.

44. Ramaurti B S, Orr T E, Bragdon C R, Lowenstein J D, Jasty M, Harris W H (1997) Factors influencing stability at the interface between a porous surface and cancellous bone: A finite element analysis of a canine in vivo micromotion experiment. J Biomed Mater Res 36:274-280.

45. Rawlinson S C F, Mosley J R, Suswillo R F L, Pitsillides A A, Lanyon L E (1995) Calvarial and limb bone cells in organ and monolayer culture do not show the same early response to dynamic mechanical strain. J Bone Min Res 10: 1225-1232.

46. Rondelli G (1996) Corrosion resistance tests on NiTi shape memory alloy. Biomaterials 17:2003-2008.

47. Sarkar N K, Redmond W, Schwaninger B, Goldberg A J (1983) The chloride behaviour of four orthodontic wires. J Oral Rehab 10:121-128.

48. Schmerling M A, Wilkov M A, Sanders A E (1976) Using the shape recovery of nitinol in the Harrington Rod treatment of scoliosis. J Biomed Mater Res 10:879-892.

49. Schwartz Z, Somers A, Mellonig J T, Carnes D L, Wozney J M, Dean D D, Cochran D L, Boyan B D (1998) Addition of human recombinant bone morphogenetic protein-2 to inactive commercial human demineralized freeze-dried bone allograft makes an effective composite bone inductive implant material. J. Periodontol 69:1337-1345.

50. Shabalovskaya S A (1996) On the nature of the biocompatibility and on the medical applications of NiTi shape memory alloys. Biomed Mater Eng 6:267-289.

51. Simske S J, Ayers R A, Bateman T A (1997) Porous materials for bone engineering. In: Liu D M, Dixit V (eds) Porous materials for tissue engineering, Trans Tech Publications, Uetikon-Zuerich, p 151.

52. Simske S J, Sachdeva R (1995) Cranial bone apposition and ingrowth in a porous nickel-titanium implant. J Biomed Mat Res 29:527-533.

53. Spector M (1987) Historical review of porous-coated implants. J Arthroplasty 2:163177.

54. Szachowicz E H (1995) Facial bone wound healing: An overview. Otolaryngol Clin North Am 28:865-880.

55. Takeshita F, Takata H, Ayukawa Y, Suetsugu T (1997) Histomorphometric analysis of the response of rat tibiae to shape memory alloy (nitinol). Biomaterials, 18:21-25.

56. Trepanier C, Tabrizian M, Yahia L H, Bilodeau L, Piron D L (1998) effect of modification of oxide layer n NiTi stent corrosion resistance. J Biomed Mater Res 43:433-440.

57. van Eeden S P, Ripamonti U (1994) Bone differentiation in porous hydroxyapatite in baboons is regulated by the geometry of the substratum: implications for reconstructive surgery. Plast Reconstr Surg 93:959-966.

58. Vercaigne S, Wolke J G C, Naert 1, Jansen J A (1998) Histomorphometrical and mechanical evaluation of titanium plasma-spray-coated implants placed in the cortical bone of goats. J Biomed Mater Res 41:41-48.

59. Villermaux F, Fabrizian M, Yahia L H, Czeremuszkin G, Piron D L (1996) Corrosion resistance improvement of NiTi osteosynthesis staples by plasma polymerized tetraflouroethylene coating. Biomed Mater Eng 6:241-254.

60. Wolford L M, Wardrop R W, Hartog J M (1987) Coralline hydroxylapatite as a bone graft substitute in orthognathic surgery. J Oral Maxillofac Surg 45:1034-1042. Yahia L H, Lombardi S, Piron D, Klemberg-Sapieha J E, Wertheimer M R (1997) NiTi shape memory alloys treated by plasma-polymerized tetraflouroethylene. Med Prog Tech 21:187-193.

62. Yi H C, Moore J J (1990) The combustion synthesis of NiTi shape memory alloys. J Minerals, Metals and Materials Society 42:31-35.

63. Agrawal C M, Best J, Heckman J D, Boyan B D (1995) Protein release kinetics of a biodegradable implant for fracture non-unions. Biomaterials 18:1255-1280.

64. Airoldi G. Riva G (1996) Innovative materials: the NiTi alloys in orthodontics. Biomed Mater Eng 6:299-305.

65. Ayers R A, Simske S J, Bateman T A, Petkus A, Sachdeva RLC, Gyunter V E (I 999a) Effect of nitinol implant porosity on cranial bone ingrowth and apposition after 6 weeks. J Biomed Mater Res 45:42-47.

66. Ayers R A, Sihiske S J, Nunes C R, Wolford L M (1998) Long-term bone ingrowth and residual microhardness of porous block hydroxyapatite in humans. J Oral Maxillofac Surg, 56:1,297-1301.

67. Ayers R A, Wolford L M, Bateman T A, Ferguson V L, Simske S J (I 999b) Quantification of bone ingrowth into porous block hydroxyapatite in humans. J Biomed Mater Res 47:54-59.

68. Bajpai P K, Billotte W G (1995) Ceramic biomaterials. In: Bronzino J D (ed) The biomedical engineering handbook, CRC Press, Boca Raton, p 552.

69. Barth E, Ronningen H, Solheim L, Sactnren B (1986) Bone ingrowth into weightbearing porous fiber titanium implants. Mechanical and biochemical correlations. J Ortho Res, 4:356-361.

70. Berger-Gorbet M, Broxup B, Rivard C, Yahia L H (1996) Blocompatibility testing of NiTi screws using immunohistochemestry on section containing metallic implants. J Biomed Mater Res 32:243-248.

71. Bragdon C R, Burke D, Lowenstein J D, O'Connor D O, Ramamurti B, Jasty M, Harris W H (1996) Differences in stiffness of the interface between a cemetless porous implant and cancellous bone in vivo in dogs due to varying amounts of implant motion. J Arthroplasty 11:945-951.

72. Broz A Simske S J, Corley W D, Greenberg A R (1997) Effects of deproteinization and ashing on site-specific properties of cortical bone. J Mater Sci Mater Med 8:395-401.

73. Castleman L S, Motzkin S M, Alicandri F P, Bonawit V L (1976) Biocompatibility of nitinol alloy as an implant material. J Biomed Mater Res 10:695-731.

74. Chang Y S, Oka M, Takashi N, Gu H O (1996) Bone remodeling around implanted ceramics. J Biomed Mater Res, 30:117-124.

75. Ciirrey J D, Brear K: Hardness (1990) Young's modulus and yield stress in mammalian mineralized tissues. J Mat Sci: Materials in Medicine, 1: 14.

76. Dai K (1996) Studies and applications of NiTi shape memory alloys in the medical field in China. Biomed Mate'r Eng 6:233-240.

77. Desilets C P, Marden L J, Patterson A L, Hollinger J O (1992) Development of synthetic materials for craniofacial reconstruction. J Craniofac Surg 1: 150-153.

78. Ducheyne P (1998) Bioactive calcium phosphate ceramics and glasses. In: Sedel L, Cabanela M E (eds) Hip surgery: Materials and developments, Mosby, St. Louis, p 75.

79. Eggli P S, Mtiller W, Scherik R K (1988) Porous hydroxyapatite and tricalcium phosphate cylinders with two different pore size ranges implanted in the cancellous bone of rabbits. A comparative histomorphometric and histologic study of bony ingrowth and implant substitution. Clin Orthop 232:127-138.

80. Endo K (1995) Chemical modification of metallic implant surfaces with biofunctional proteins (part 1) molecular structure and biological activity of a modified NiTi alloy surface. J Dent Mater 14:185-198.

81. Endo K (1995) Chemical modification of metallic implant surfaces with biofunctional proteins (part 2) corrosion resistance of a chemically modified NiTi alloy. J Dent Mater 14:199-210.

82. Engh C A, Bugbee W D (1998) Extensively porous-coated femoral stems. In: Sedel L, Cabanela M E (eds) Hip surgery: Materials and developments, Mosby, St. Louis, p 243.

83. Eppley B L, Sadove A M, (1990) Effects of material porosity on implant bonding strength in a craniofacial model. J Craniofac Surg 1: 191-195.

84. Gao T i, Lindholm T S, Konimonen B, Ragni P, Paronzini A, Lindholm T C, Jalovaara P, Urist M R (1997) The use of a coral composite implant containing bone morphogenetic protein to repair a segmental tibial dzfect in sheep. Int Orthoped 21:194-200.

85. Goldberg R A, Garbutt M, Shorr N (1993) Oculoplastic uses of cranial bone grafts. Ophalmic Surg, 24:190-196.

86. Greene D, Pruitt L, Maas C S (1997) Bipmechanical effects of e-PTFE implant structure on soft tissue implantation stability: a study in the porcine model. Laryngoscope 107:957-962.

87. Guicheux J, Gauthier 0, Aguado E, Heymann D, Pilet P, Couillaud S, Faivre A, Daculsi G (1998) Growth hormone-loaded marcroporous calcium phosphate ceramic: in vitro biopharmaceutical characterization and preliminary in vivo study. J Biomed Mater Res 24:47-63.

88. Hiatt W R, Moore D L, Mosby E L, Fain D W (1987) Preformed hydroxylapatite blocks for palatal grafting in orthognathic surgery. Int. J Oral Maxillfac Surg, 16:302-304.

89. Hofmann A, Bloebaum R D, Bachus K N (1997) Progression of human bone ingrowth into porous coated implants. Acta Orthop Scand, 68:161-166.

90. Hollinger J (1993) Strategies for regenerating bone of the craniofacial complex. Bone 14:575-580.

91. Hollister S J, Kikuchi N, Goldstein S A (1993) Do bone ingrow-th processes produce a globally optimized structure? J Biomech 26:391-407.

92. Holmes R E, Wardrop R W, Wolford L M (1988) Hydroxylapatite as bone graft substitute in orthognathic surgery: histologic and histometric findings. J Oral Maxillofac Surg 46:661-671.

93. Houde J, Marchetti M, Duquette J, Hoffman A, Steinberg G, Crane K, Baran D (1995) Correlation of bone mineral density and femoral neck hardness in bovineand human samples. Calcif Tissue Int, 57: 201-205.

94. Hulbert S F, Young F A, Mathews R S, Klawitter J J, Talbert CD Stelling FH (1970) Potential of ceramic materials as permanently implantable skeltal prostheses. J Biomed Mater Res 4:433-456.

95. Itin V I, Gyunter V E, Shabalovskaya S A, Sachdeva RLC (1994) Mechanical properties and shape memory of porous nitinol. Materials Characterization 32:179-187.

96. Itin V I, Shevcheako N A, Korosteleva, Tukhfatullin A A, Mirgazizov M Z, Gyunter V E (1997) “Bioceramic-titanium nickelide” functional composites for medicine. Tech Phys Lett 23:294-296.

97. Ivanoff C J, Sennerby L, Lekholm U (1997) Reintegration of mobilized titanium implants: an experimental study in rabbit tibia. J Oral Maxillofac Surg, 26:3 10-315.

98. Jalul A F (1992) Experimental applications of porous (coralline) hydroxylapatite in middle ear and mastoid reconstruction. Laryngoscope 102:289-299.

99. Kent J N, Zide M F (1984) Wound healing: bone and biomaterials. Otolarynogol Clin North Am 17:273-319.

100. Kieswerter K, Schwartz Z, Dean D D, Boyan B D (1996) The role of implant surface characteristics in the healing of bone. Crit Rev Oral Biol Med 7:329-3-45.

101. Klawitter J J, Hulbert S F (197 1) Application of porous ceramics for the attaclunent of load bearing internal orthopedic applications, J Biomed Mater Res Symp 2:161-229.

102. Klinge B, Alberius P, Isaksson S, Jonsson J (1992) Osseous response to implanted natural bone mineral and synthetic bydroxylapatite ceramic in the repair of experimental skull bone defects. J Oral Maxillofac Surg, 50:241-249.

103. Lakes R (1995) Composite biomaterials. In: Bronzino J D (ed) The biomedical engineering handbook, CRC Press, Boca Raton, p 598.

104. Light M, Kanat 10 (199 1) The possible use of coralline hydroxyapatite as a bone implant. J Foot Surg 30:472-476.

105. Martin R B, Chapman M W, Holmes R E, Sartoris D J, Shors E C, Gordon J E, Heitter D O, Sharkey N A, Zissimos A G (1989) Effects of bone ingrowth on the strength and non-invasive assessment of a coralline hydroxyapatite material. Blomaterials 10:481-488.

106. Martin R B, Chapman M W, Sharkey N A, Zissimos A G, Bay B, Shors EC (1993) Bone ingrowth and mechanical properties of coralline hydroxyapatite I yr after implantation. Biomaterials 14:341-348.

107. Melton KN and Harrison J D (1994) Corrosion of Ni-Ti based shape memory alloys. Proc. Int Conf Shape Memory Superelastic Tech 1: 187-196.

108. Meng-Hai B, Xing-Yan L, Bao-Feng G, Chao Y, Dong-An C (1996) An implant of a composite of bovine bone morphogenetic protein and plaster of paris for treatment of femoral shaft nonunions. Int Surg 81:390-392.

109. Motoki D S, Mulliken J B (1990) The healing of bone and caililage. Clin Plast Surg, 17:527-544.

110. Nelson S R, Wolfo7d L M, Lagow R J, Capano P J, Davis VVL (1993) Evaluation of new high perforinance calcium polyphosphate bioceramics as bone graft materials. J Oral Maxillofac Surg 51: 1363-1371.

111. Neo M, Voigt C F, Herbst H, Gross U M (1998) Osteoblast activity at the interface between surface-active materials and bone in uivo: a study using in situ hybridization. J Biomed Mater Res 39:1-8.

112. Nunes C R, Simske S J, Sachdeva R, Wolford L M (1997) Long-term. ingrowth and apposition of porous hydroxylapatite implants. J Biomed Mater Res 36:560-563.

113. Oberg S, Rosenquist J B (1994) Bone healing after implantation of hydroxyapatite granules and blocks (Interpore 200®) combined with autolyzed antigen-extracted allogenic bone and fibrin glue. Int J Oral Maxillofac Surg, 23:110-114.

114. Ono K, Yamamuro T, Nakamura T, Kokubo T (1990) Mechanical properties of bone after implantation oPapatite-wollastonite containing glass ceramic-fibrin mixture. J Biomed Mater Res 24:47-63.

115. Oshida. Y, Sachdeva R, Miyazaki S (1992) Changes in contact angles as a function of time on some pre-oxidized biomaterials. J Mater Sci Mater Mcd 3:306-312.

116. Otsuka K, Wayman C M (1998) Mechanism of shape memory effect and superelasticity. In: Otsuka K, Wayman C M (eds) Shape memory materials, Cambridge University Press, Cambridge, p 27.

117. Parfitt A M, Drezner M K, Glorieux F H, Kanis J A, Malluche H, Meunier P J, Ott S M, Recker R R (1987) Bone histomorphometry: Standardization of nomenclature, symbols, and units. J Bone Min Res 2:595-610.

118. Parfitt AM: Stereologic basis of bone histomorphometry; Theory of quantitative microscopy and reconstruction of the third dimension, in Recker H R (ed) Bone Histomorphometry: Techniques and Interpretation. Boca Raton, Fla., CRC Press, 1983, pp 53-87.

119. Parfitt AM: The physiologic and clinical significance of bone histomorphometric data, in Recker FIR (ed) Bone Histomorphometry: Techniques and Interpretation. Boca Raton, Fla., CRC Press, 1983, pp 143-217.

120. Pedersen D R, Brown T D, Brand R A (199 1) Interstial bone stress distributions accompanying ingrowth of a screen-like prosthesis anchorage layer. J Biomech 24: 1131-1142.

121. Phillips J H, Forrest C R, Gruss J S (1992) Current concepts in the use of bone grafts in facial fractures. Clin Plast Surg 19:41-58.

122. Prendergast P J, Huiskes R, Soballe K (1997) Biophysical stimuli on cells during tissue differentiation at implant interfaces. J Biomechanics, 30:539-548.

123. Putters J L M, Kaulesar Sukul DMKS, deZeeuw G R, Bijma A, Besselink Pa (1992) Comparative cell culture effects of shape memory metal (Nitinol), nickel and titanium: A biocoliipatibility estimation. Eur Surg Res 24:378-382.

124. Ramaurti B S, Orr T E, Bragdon C R, Lowenstein J D, Jasty M, Harris WH (1997) Factors influencing stability at the interface between a porous surface and cancellous bone: A finite element analysis of a canine in vivo micromotion experiment. J Biomed Mater Res 36:274-280.

125. Rawlinson S C F, Mosley J R, Suswillo R F L, Pitsillides A A, Lanyon L E (1995) C˜lvarial and limb bone cells in organ and monolayer culture do not show the sanie early response to dynamic mechanical strain. J Bone Min Res 10: 1225- i 232.

126. Riparrionti U (199 1) The induction of bone in osteogenic composites of bone matrix and porous hydroxyapatite replicas: An experimental study on the baboon (Papio ursinus). J Oral Maxillofac. Surg 49:817-830.

127. Riparrionti U, Petit J C, Moehl T, van den Heever B, van Wyk J (1993) Immediate reconstruction of massive cranio-orbito-facial defects with allogenic and alloplastic matrices in baboons. J Cranio Maxillo Fac, Surg 21:302-308.

128. Rondelli G (1996) Corrosion resistance tests on NiTi shape memory alloy. Biomaterials 17:2003-2008.

129. Sarkar N K, Redmond W, Schwaninger B, Goldberg A J (1983) The chloride behaviour of four orthodontic wires. J Oral Rehab 10:121-128.

130. Schenk R K, Olah A J, Herrmann: Preparation of calcified tissues for light microscopy, in Dickson, G R (ed) Methods of Calcified Tissue Preparation, New York, Elsevier Science, 1984, 1997, pp. 1-56.

131. Schmerling M A, Wilkov M A, Sanders A E (1976) Using the shape recovery of nitinol in the Harrington Rod treatment of scoliosis. J Biomed Mater Res 10:879892.

132. Schwartz Z, Somers A, Mellonig J T, Carries D L, Wozney J M, Dean D D, Cochran D L, Boyan B D (1998) Addition of human recombinant bone morphogenetic protein-2 to inactive commercial human demineralized freeze-dried bone allograft makes an effective composite bone inductive implant material. J Periodontol 69:1337-1345.

133. Shabalovskaya S A (1996) On the nature of the biocompatibility and on the medical applications of NiTi shape memory alloys. Biomed Mater Eng 6:267-289.

134. Shabalovskaya S A, Itin V I, Gyunter V E (1994) Porous Ni-Ti - A new material for implants and prostheses. Proc Int Conf Shape Memory Superelastic Tech 1:7-12.

135. Simske S J, Ayers R A, Bateman T A (1997) Porous materials for bone engineering. In: Liu D M, Dixit V (eds) Porous rn′ aterials for tissue engineering, Trans Tech Publications, Uetikon-Zuerich, p 151.

136. Simske S J, Broz J J, Luttges M W (1995) Effect of suspension on mouse bone microhardness. J Mat Sci Mat in Med 6:486.

137. Simske S J, Sachdeva R (1995) Cranial bone apposition and ingrowth in a porous nickel-titanium implant. J Biomed Mat Res 29:527-533.

138. Spector M (1987) Historical review of porous-coated implants. J Arthroplasty, 2:163-177.

139. Spector M, “Bone growth into porous metals,” in Biocompatibility of Orthopedic Iniplants, vol. 2, D. F. Williams (ed.), Boca Raton, Fla., CRC Press, Boca Raton, Fla., 1982, pp. 55-88.

140. Sysolyatin P G, Gyunter V E, Starokha A V, Makarova I A, Sysolyatin S P, Denisov V N, Rahman B Q (1994) The use of Ni-Ti implants in maxillofacial surgery. Proc Int Conf Shape Memory Superelastic Tech 1:471-475.

141. Szachowicz E H (1995) Facial bone wound healing: An overview Otolaryngol Clin North Am 28:865-880.

142. Takeshita F, Takata H, Ayukawa Y, Suetsugu T (1997) Histomorphometric analysis of the response of rat tibiae to shape memory alloy (nitinol). Biomaterials, 18:21-25.

143. Trepanier C, Tabrizian M, Yahia L H, Bilodeau L, Piron D L (1998) effect of modification of oxide layer n NiTi stent corrosion resistance. J Biomed Mater Res 43:433-440.

144. van Eeden S P, Ripamonti U (1994) Bone differentiation in porous hydroxyapatite in baboons is regulated by the geometry of the substratum: implications for reconstructive surgery. Plast Reconstr Surg 93:959-966.

145. Vercaigne S, Wolke JGC, Naert I, Janseii J A (1998) Histomorphometrical and mechanical evaluation of titanium plasma-spray-coated implants placed in the cortical bone of goats. J Biomed Mater Res 41:41-48.

146. Villermaux F, Tabrizian M, Yahia L H, Czeremuszkin G, Piron D L (1996) Corrosion resistance improvement of NiTi osteosynthesis staples by plasma polymerized tetraflouroethylene coating. Biomed Mater Eng 6:241-254.

147. Wolford L M, Wardrop R W, Hartog J M (1987) Coralline hydroxylapatite as a bone graft substitute in orthognathic surgery. J Oral Maxillofac Surg 45:10341042.

148. Yahia L H, Lombardi S, Piron D, Klemberg-Sapieha J E, Wertheimer MR (1997) NiTi shape memory alloys treated by plasma-polymerized tetraflouroethylene. Med Prog Tech 21:187-193.

149. Yi H C, Moore J J (1990) The combustion synthesis of NiTi shape memory alloys. J Minerals, Metals and Materials Society 42:3) 1-35.

The embodiments of the invention in which an exclusive property or privilege is claimed are defined as follows:

1. A nonuniform porosity tissue implant comprising, a porous biomaterial having a nonrandom functionally graded porosity which mimics a whole bone cross-section.
